(19) United States
(12) Patent Application Publication
(10) Pub. No.: US 2023/0129798 A1
(43) Pub. Date: Apr. 27, 2023

(54) Nasal Ventilation Mask

(71) Applicants: Derek Lawrence Isenberg, Haddon Heights, NJ (US); James Wittes, Princeton, NJ (US); Philip J. Blyskal, Belmont, VT (US)

(72) Inventors: Derek Lawrence Isenberg, Haddon Heights, NJ (US); James Wittes, Princeton, NJ (US); Philip J. Blyskal, Belmont, VT (US)

(21) Appl. No.: 18/045,379

(22) Filed: Oct. 10, 2022

Related U.S. Application Data
(63) Continuation-in-part of application No. 17/814,602, filed on Jul. 25, 2022.
Application No. 17/814,602 is a continuation of application No. 17/356,289, filed on Jun. 23, 2021, now Pat. No. 11,426,551.
(60) Provisional application No. 63/175,774, filed on Apr. 16, 2021.
(60) Provisional application No. 63/152,452, filed on Feb. 23, 2021.
(60) Provisional application No. 63/042,896, filed on Jun. 23, 2020.

(51) Int. Cl. A61M 16/06; A61M 16/00

(52) U.S. Cl. CPC A61M 16/0616; A61M 16/0084; A61M 2210/0618

(57) ABSTRACT

A nasal ventilation mask supplies breathing gas to an airway of a patient. The mask has a housing defining an internal cavity and having a peripheral end defining an end opening. The end opening is in fluid communication with the internal cavity, and the end opening is configured to be positioned over an airway on a face of a patient. The housing further defines a connector segment having a receiver member. The connector member defines a top wall defining an outer periphery. The receiver member has a portion extending past the outer periphery. The receiving member has an opening in fluid communication with the internal cavity. The mask also has a sealing member operably connected to the housing proximate the end opening. The sealing member is configured to abut the face of the patient, and the receiver member is configured to be operably connected to a supply of breathing gas.

### CROSS-REFERENCE TO RELATED APPLICATIONS

The present application is a continuation-in-part patent application of U.S. patent application Ser. No. 17/814,602, filed on Jul. 25, 2022, which is a continuation patent application of U.S. patent application Ser. No. 17/356,289, filed on Jun. 23, 2021, now U.S. Pat. No. 11,426,551, which claims the benefit of U.S. Provisional Patent Application No. 63/175,774, filed on Apr. 16, 2021, U.S. Provisional Patent Application No. 63/152,452, filed on Feb. 23, 2021, and U.S. Provisional Patent Application No. 63/042,896, filed on Jun. 23, 2020.

### FEDERALLY SPONSORED RESEARCH OR DEVELOPMENT

None.

### TECHNICAL FIELD

The invention relates generally to a respiratory mask and, in particular, a nasal ventilation mask for use with a manual resuscitator for ventilation of a patient.

### BACKGROUND OF THE INVENTION

Manual resuscitators, or manual resuscitator bag assemblies, or artificial manual breathing units are known in the art and used in medical settings. These devices are sometimes referred to as bag valve masks or breathing units as well. Artificial manual breathing units and manual resuscitator bag assemblies incorporate a face mask to cover the nose and/or mouth of a patient for ventilation (supplying breathing gas to lungs of a patient). Trained medical personnel such as doctors, nurses, paramedics or emergency medical technicians, resuscitators or other emergency responders use the breathing unit as a medical tool to supply breathing gas to an airway of a patient, e.g., to force air into the lungs of the patient who is not breathing or is not breathing adequately and needs assistance.

As shown in FIGS. 1 and 2, such manual resuscitators, designated with the reference numeral 1, typically include a squeezable, inflatable bag 2, and a face mask 3 operably connected to the bag 2. FIGS. 1 and 2 show prior art manual resuscitator bag assemblies. In addition to being connected to the face mask 3, the bag 2 can also have a tube(s) that can be connected to an oxygen source to be used if desired. The face mask 3 may have an inflatable seal portion 4 designed to be pressed against the patient's face when the face mask 3 is placed over the patient's mouth and nose. FIG. 2 shows a medical worker using the bag assembly 1 on a patient and squeezing the inflatable bag 2 to force air into the lungs of the patient. Emergency responders are trained in the use of the manual resuscitator devices as further injury or even death to the patient can result if the device not used properly.

In many manual resuscitator bag assembly designs, it is difficult for medical personnel to achieve a tight seal against the patient's face. Certain mask designs make it difficult for medical personnel or emergency responders to engage the mask to assist in creating a tight seal. In certain mask design structures, responders experience difficulty in pressing the mask towards a patient while attempting to engage the patient for additional support. These designs are not ergonomically efficient for most responders. In certain uses, multiple workers may be required to successfully use the device. A less than tight seal prevents proper operation of the manual resuscitator. In other designs, the face masks have more complex multi-component designs that are more rigid, costly and cumbersome to use. Other face masks are designed to cover both a nasal airway and an oral airway resulting in other difficulties if medical personnel require access to the oral airway while still being able to deliver oxygen to the nasal airway.

While such face masks used with manual resuscitators according to the prior art provide a number of advantageous features, they nevertheless have certain limitations. The present invention is provided to overcome certain of these limitations and other drawbacks of the prior art, and to provide new features not heretofore available. A full discussion of the features and advantages of the present invention is deferred to the following detailed description, which proceeds with reference to the accompanying drawings.

### SUMMARY OF THE INVENTION

The present invention provides a nasal ventilation mask for use with an artificial manual breathing unit or manual resuscitator bag assembly for ventilation of a patient.

According to a first aspect of the invention, a ventilation mask for supplying breathing gas to an airway of a patient has a housing defining an internal cavity. The housing also has a peripheral end defining an end opening, the end opening being in fluid communication with the internal cavity. The end opening is configured to be positioned over an airway on a face of a patient. The housing further has a connector segment having a receiver member. The connector segment defines a top wall defining an outer periphery wherein the receiver member has a portion extending past the outer periphery. The receiving member has an opening in fluid communication with the internal cavity. A sealing member is operably connected to the housing proximate the end opening, and the sealing member is configured to abut the face of the patient. The receiver member is configured to be operably connected to a supply of breathing gas.

According to another aspect of the invention, the top wall defines a central portion. The receiver member extends from the top wall at a location remote from the central portion.

According to another aspect of the invention, the housing defines an upper sidewall segment. The receiver member has a portion extending from the top wall and a portion extending from the upper sidewall segment.

According to a further aspect of the invention, the housing defines a front wall section and a rear wall section depending from the top wall. The receiver member has a portion extending from the top wall and a portion extending from the rear wall section

According to yet a further aspect of the invention, the housing defines a front wall section and a rear wall section depending from the top wall. The housing defines a nose slot in the front wall section. The receiver member extends from the top wall proximate the rear wall section and generally opposite the nose slot.

According to a further aspect of the invention, the housing defines a front wall section and a rear wall section depending from the top wall. The housing defines a nose slot in the front wall section. The receiver member defines a cylindrical wall wherein a portion of the cylindrical wall extends from the top wall and a portion of the cylindrical wall extends from the rear wall section and generally opposite the nose slot.

According to another aspect of the invention, the top wall defines an outer periphery and a center point location within the outer periphery. The receiver member extends from the top wall at a location remote from the center point location and proximate the outer periphery.

According to yet another aspect of the invention, the opening of the receiver member is not concentric with the end opening. In an exemplary embodiment, the receiver member is integral with the connector segment.

According to another aspect of the invention, the sealing member has a reinforcing member extending from the housing. The housing defines an upper sidewall segment depending from the top wall. The receiver member has a portion extending from the top wall and a portion extending from the upper sidewall segment that is generally aligned with an end of the reinforcing member.

According to another aspect of the invention, the sealing member extends towards the internal cavity. The sealing member forms an inwardly curled lip member extending into the internal cavity, and the lip member having an outer surface configured to abut the face of the patient. The lip member has a proximal end connected at the peripheral end, and the lip member having a distal end. The lip member has a thickness being tapered towards the distal end. The housing defines an internal surface wherein an imaginary line extends from the internal surface past the end opening, wherein a distal end of the lip member extends past the imaginary line. An outer surface of the inwardly curled lip has a convex shape and is smooth and uninterrupted.

According to another aspect of the invention, the housing has a reinforcing member extending therefrom proximate the end opening. The reinforcing member has an outer end. The sealing member defines an inwardly curled lip member having a proximal end depending from the outer end of the reinforcing member. In one exemplary embodiment, the housing defines an internal surface wherein an imaginary line extends from the internal surface past the end opening. A distal end of the lip member extends past the imaginary line.

According to yet another aspect of the invention, the housing further defines a housing body segment operably connected to the connector segment defining a circumferential connection joint. The housing defines a front wall section opposite a rear wall section and a first end wall section extending between the front wall section and the rear wall section. The connection joint extends downwardly along a contoured path on the first end wall section from the front wall section towards the rear wall section. In an exemplary embodiment, the connection joint on the first end wall section has a curved segment and a substantially horizontal segment proximate the rear wall section.

According to another aspect of the invention, the housing body segment defines a lower sidewall segment and the connector segment defines an upper sidewall segment. The upper sidewall segment is positioned inwardly offset from the lower sidewall segment. The lower sidewall segment has a first thickness and the upper sidewall segment has a second thickness, the first thickness being greater than the second thickness.

According to another aspect of the invention, the connection joint is formed from confronting connection surfaces between the housing body segment and the connector segment. A chemical bond is formed between the materials of the housing body segment and the connector segment

According to a further aspect of the invention, the housing body segment is of a thermoplastic elastomer material. Also, the connector segment is of an acrylonitrile butadiene styrene material.

According to another aspect of the invention, the top wall has a gripping member thereon. In an exemplary embodiment, the gripping member comprises a plurality of protrusions. In a further exemplary embodiment, the top wall has a curved configuration from a first top wall end to a second top wall end.

According to another aspect of the invention, the receiver member extends generally perpendicular from the top wall. When the mask is configured to abut the face of the patient, the receiver member is configured to be generally perpendicular to the face of the patient

According to another aspect of the invention, a ventilation mask for supplying breathing gas to an airway of a patient has a housing defining an internal cavity and having a peripheral end defining an end opening. The end opening is in fluid communication with the internal cavity, and the end opening configured to be positioned over an airway on a face of a patient. The peripheral end of the housing has an inwardly curled lip extending towards the internal cavity. The inwardly curled lip is configured to abut the face of the patient and deflect towards the internal cavity. The housing further defines a front wall section, a rear wall section and opposite end wall sections extending between the front wall section and the rear wall section. The housing has a housing body segment operably connected to a connector segment defining a circumferential connection joint around the housing. On opposite end wall sections, the connection joint has segments sloping downwardly along a curved path from the front wall section towards the rear wall section. Further on the opposite end wall sections, the connection joint has horizontal segments at the rear wall section and extending from the rear wall section towards the front wall section and joining the segments sloping downwardly. The connector segment defines a top wall defining a central portion. The connector segment further has a receiver member extending from the top wall at a location remote from the central portion. The receiving member has an inlet opening in fluid communication with the internal cavity, and the receiving member is configured to be operably connected to a supply of breathing gas.

According to yet another aspect of the invention, a nasal ventilation mask for supplying breathing gas to an airway of a patient has a housing. The housing has a front wall section, a rear wall section and opposite end wall sections extending between the front wall section and the rear wall section cooperatively defining an internal cavity and having a peripheral end defining an end opening. The housing further defines a nose slot in the front wall section, the nose slot being in fluid communication with the end opening. The end opening is in fluid communication with the internal cavity. The end opening is configured to be positioned over an airway on a face of a patient with the nose slot configured to be positioned over a nose of the patient. The housing further defines a connector segment defining a top wall defining an outer periphery. The connector segment further has a receiver member defining an inlet opening in communication with the internal cavity wherein the receiver member extends from the top wall and the rear wall section generally opposite the nose slot in the front wall section wherein a portion of the receiver member extends past the outer periphery of the top wall. A sealing member is operably connected to the housing proximate the end opening, and the sealing member is configured to abut the face of the patient. The receiver member is configured to be operably connected to a supply of breathing gas.

According to another aspect of the invention, a nasal ventilation mask for supplying breathing gas to a nasal airway of a patient has a housing defining an internal cavity. The housing has a housing body segment of flexible material operably connected to a connector segment of rigid material defining a circumferential connection joint. The housing body segment defines a distal end defining an end opening in fluid communication with the internal cavity. The housing body segment further defines a nose slot on a side of the housing wherein the noses slot is generally transverse to the end opening, the slot being in fluid communication with the end opening. The distal end has a sealing member positioned around the end opening and slot, and the sealing member forms an inwardly curled lip member extending into the internal cavity. The connector segment defines a top wall connected to an upper sidewall segment. The top wall defines an outer periphery and has a plurality of rigid protrusions extending from the top wall. The connector segment further has a receiver member defining an inlet opening in communication with the internal cavity wherein the receiver member extends from the top wall and the upper side segment generally opposite the nose slot wherein a portion of the receiver member extends past the outer periphery of the top wall. The top wall defines a contiguous uninterrupted surface area across a central portion of the top wall for applying a pressing force thereon. The top wall is configured to be pressed by a responder towards a face of the patient wherein the lip member is configured to engage the face of the patient and deflect into the internal cavity and configured to create a seal between the face of the patient and the lip member, and wherein the slot is dimensioned to be positioned proximate a dorsal base of a nose of the patient. An inflatable bag of a manual resuscitator bag assembly is configured to be operably connected to the inlet opening of the receiver member of the connector segment such that breathing gas is configured to be delivered to the nasal airway of the patient.

According to yet another aspect of the invention, a ventilation mask for supplying breathing gas to an airway of a patient has a housing defining an internal cavity and having a peripheral end defining an end opening. The end opening is in fluid communication with the internal cavity, and the end opening is configured to be positioned over an airway on a face of a patient. The housing further has a top wall and a sidewall depending from the top wall at a connection interface. The top wall has an extension wall member extending from the connection interface. The housing also has a receiver member extending from the top wall. The receiving member has an opening in fluid communication with the internal cavity. A sealing member is operably connected to the housing proximate the end opening, and the sealing member is configured to abut the face of the patient. The receiver member is configured to be operably connected to a supply of breathing gas.

According to another aspect of the invention, the top wall defines a central portion. The receiver member extends from the top wall at a location remote from the central portion.

According to another aspect of the invention, the top wall defines an outer periphery wherein the receiver member is positioned on the top wall at the outer periphery.

According to another aspect of the invention, a plurality of support members are positioned between an underside surface of the extension wall member and the sidewall.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a perspective view of a prior art manual resuscitator bag assembly;

FIG. 2 is a schematic side elevation view of a medical worker using a prior art manual resuscitator bag assembly on a patient;

FIG. 3 is a front perspective view of a nasal ventilation mask according to an exemplary embodiment of the present invention;

FIG. 4 is a rear perspective view of the nasal ventilation mask of FIG. 3;

FIG. 5 is a front elevation view of the nasal ventilation mask of FIG. 3;

FIG. 6 is a rear elevation view of the nasal ventilation mask of FIG. 3;

FIG. 7 is a side elevation view of the nasal ventilation mask of FIG. 3, the opposite side elevation view being the same;

FIG. 8 is a top plan view of the nasal ventilation mask of FIG. 3;

FIG. 9 is a bottom plan view of the nasal ventilation mask of FIG. 3;

FIG. 10 is a cross-sectional view taken along lines 10-10 of FIG. 8;

FIG. 11 is a cross-sectional view taken along lines 11-11 of FIG. 8;

FIG. 12 is a front perspective view of a housing body segment of the nasal ventilation mask of FIG. 3;

FIG. 13 is a rear perspective view of a housing body segment of the nasal ventilation mask of FIG. 3;

FIG. 14 is a front elevation view of the housing body segment of the nasal ventilation mask of FIG. 3;

FIG. 15 is a rear elevation view of the housing body segment of the nasal ventilation mask of FIG. 3;

FIG. 16 is a side elevation view of the housing body segment of the nasal ventilation mask of FIG. 3, the opposite side elevation view being the same;

FIG. 17 is a top plan view of the housing body segment of the nasal ventilation mask of FIG. 3;

FIG. 18 is a bottom plan view of the housing body segment of the nasal ventilation mask of FIG. 3;

FIG. 19 is a cross-sectional view taken along lines 19-19 of FIG. 17;

FIG. 20 is a cross-sectional view taken along lines 20-20 of FIG. 17;

FIG. 21 is a partial enlarged cross-sectional view of a distal end of the housing body segment of the nasal ventilation mask of FIG. 3;

FIG. 22 is a partial enlarged cross-sectional view of the distal end of the housing body segment as shown in FIG. 21 and showing additional detail;

FIG. 23 is a further enlarged cross-sectional view shown in FIG. 22;

FIG. 24 is a front perspective view of a connector segment of the nasal ventilation mask of FIG. 3;

FIG. 25 is a rear perspective view of the connector segment of the nasal ventilation mask of FIG. 3;

FIG. 26 is a front elevation view of the connector segment of the nasal ventilation

FIG. 27 is a rear elevation view of the connector segment of the nasal ventilation mask of FIG. 3;

FIG. 28 is a side elevation view of the connector segment of the nasal ventilation mask of FIG. 3, the opposite side elevation view being the same;

FIG. 29 is a top plan view of the connector segment of the nasal ventilation mask of FIG. 3;

FIG. 30 is a bottom plan view of the connector segment of the nasal ventilation mask of FIG. 3;

FIG. 31 is a cross-sectional view taken along lines 31-31 of FIG. 29;

FIG. 32 is a cross-sectional view taken along lines 32-32 of FIG. 29;

FIG. 33 is an exploded perspective view of the nasal ventilation mask of FIG. 3 and showing the housing body segment and the connector segment;

FIG. 34 is an enlarged partial cross-sectional view of the nasal ventilation mask showing a connection joint between the housing body segment and the connector segment of FIG. 3;

FIG. 35 is an enlarged partial view of the nasal ventilation mask showing an alternative connection joint between the housing body segment and the connector segment;

FIG. 36 is an enlarged partial view of the nasal ventilation mask showing another alternative connection joint between the housing body segment and the connector segment;

FIG. 37 is an enlarged partial view of the nasal ventilation mask showing another alternative connection joint between the housing body segment and the connector segment;

FIG. 38 is a schematic perspective view of the nasal ventilation mask of FIG. 3 positioned on the face of a patient;

FIG. 39 is a perspective view of the nasal ventilation mask operably connected to the manual resuscitator bag assembly and in use on a patient;

FIG. 40 is a perspective view of the nasal ventilation mask operably connected to the manual resuscitator bag assembly and in use on a patient;

FIG. 41 is a cross-sectional side elevation view of the nasal ventilation mask positioned on the face of a patient;

FIG. 42 is a cross-sectional side elevation view of the nasal ventilation mask positioned on the face of the patient and pressed against the face of the patient;

FIG. 43 is an enlarged cross-sectional side view of the nasal ventilation mask positioned on and pressed against the face of the patient;

FIG. 44 is a partial perspective view of the nasal ventilation mask positioned on the face of the patient in an alternative method;

FIG. 45 is a partial perspective view of the nasal ventilation mask positioned on the face of the patient in an alternative method;

FIG. 46 is a cross-sectional side elevation view of the nasal ventilation mask positioned on the face of a patient;

FIG. 47 is a cross-sectional side elevation view of the nasal ventilation mask positioned on the face of the patient and pressed against the face of the patient;

FIG. 48 is an enlarged cross-sectional side view of the nasal ventilation mask positioned on and pressed against the face of the patient;

FIG. 49 is a schematic plan view of a face of a patient designating portions of a nose of the patient;

FIG. 50 is a comparison view of side elevation views of the embodiments of the nasal ventilation masks of the present invention;

FIG. 51 is a top plan view of the mask of FIG. 3 showing certain dimensions associated with the mask according to exemplary embodiments of the invention;

FIG. 52A is a front elevation view of the mask of FIG. 3 showing certain dimensions associated with the mask according to exemplary embodiments of the invention;

FIG. 52B is another front elevation view of the mask of FIG. 3 showing certain dimensions associated with the mask according to exemplary embodiments of the invention;

FIG. 53 is a bottom plan view of the mask of FIG. 3 showing certain dimensions associated with the mask according to exemplary embodiments of the invention;

FIG. 54 is a cross-sectional view of the mask of FIG. 3 showing certain dimensions associated with the mask according to exemplary embodiments of the invention;

FIG. 55 is a front perspective view a nasal ventilation mask according to another exemplary embodiment of the present invention;

FIG. 56 is a top plan view of the nasal ventilation mask of FIG. 55

FIG. 57 is a bottom plan view of the nasal ventilation mask of FIG. 55

FIG. 58 is a front elevation view of the nasal ventilation mask of FIG. 55;

FIG. 59 is a side elevation view of the nasal ventilation mask of FIG. 55, the opposite side elevation view being the same;

FIG. 60 is a rear elevation view of the nasal ventilation mask of FIG. 55;

FIG. 61 is a front perspective view of the nasal ventilation mask of FIG. 3 and having a breathing attachment connected thereto;

FIG. 62 is a bottom plan view of the nasal ventilation mask having a sensor attached thereto.

FIG. 63 is front elevation of the nasal ventilation mask having an oxygen port incorporated therein; and

FIG. 64 is front elevation of the nasal ventilation mask having an oxygen port incorporated therein in an alternative configuration.

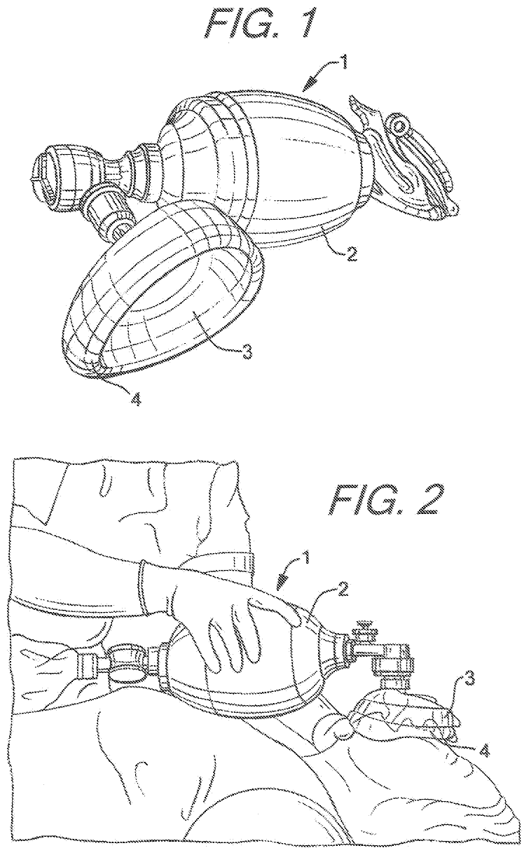

### DETAILED DESCRIPTION OF THE PREFERRED EMBODIMENTS

While this invention is susceptible of embodiments in many different forms, there are shown in the drawings and will herein be described in detail preferred embodiments of the invention with the understanding that the present disclosure is to be considered as an exemplification of the principles of the invention and is not intended to limit the broad aspect of the invention to the embodiments illustrated.

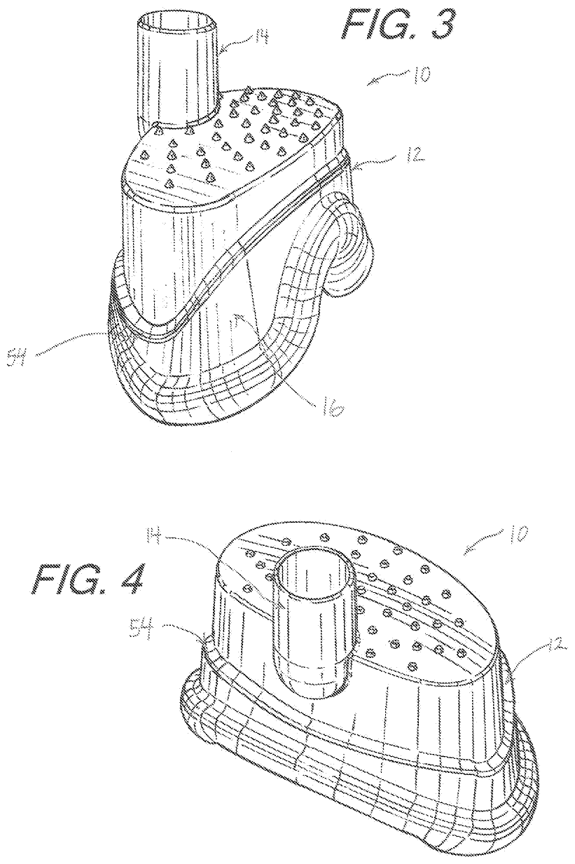

Referring to the drawings, FIG. 3 shows a perspective view of a nasal ventilation mask according to an exemplary embodiment of the invention. It is understood that the nasal ventilation mask is used as part of a manual resuscitator device to deliver a supply of breathing gas such as oxygen to a patient as described above. The structure of the nasal ventilation mask will be described followed by a description of operation of the nasal ventilation mask on a patient such as by an emergency responder, resuscitator, operator, user or other medical worker. It is further understood that the nasal ventilation mask may be referred to as a nasal mask, face mask, respiratory mask, ventilation mask or other apparatus for supplying breathing gas. It is further understood that features of the mask can apply to other breathing-type masks. Features of the mask can be used in masks designed to cover both an oral airway and nasal airway or just one airway.

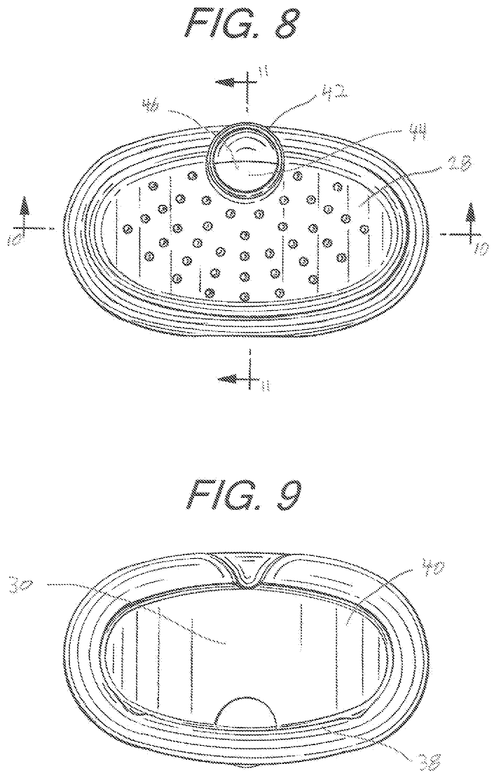

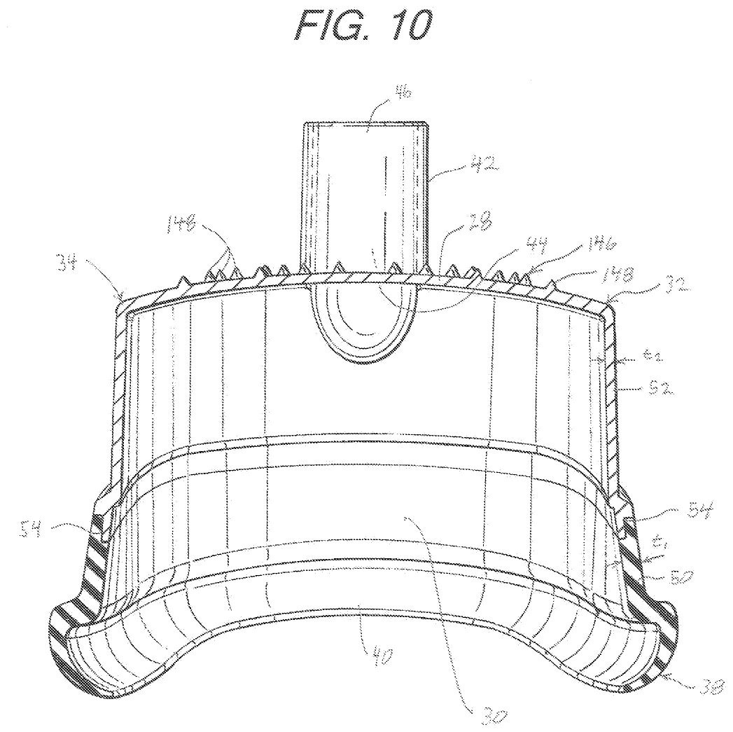

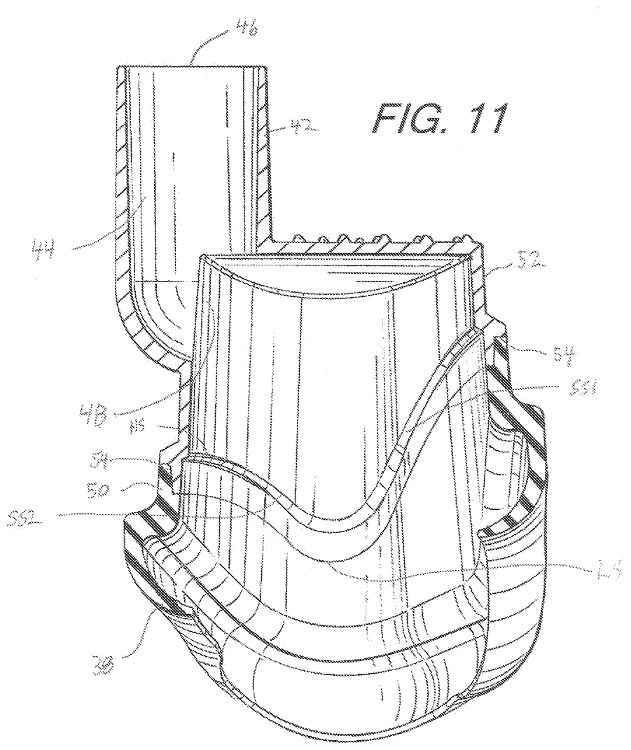

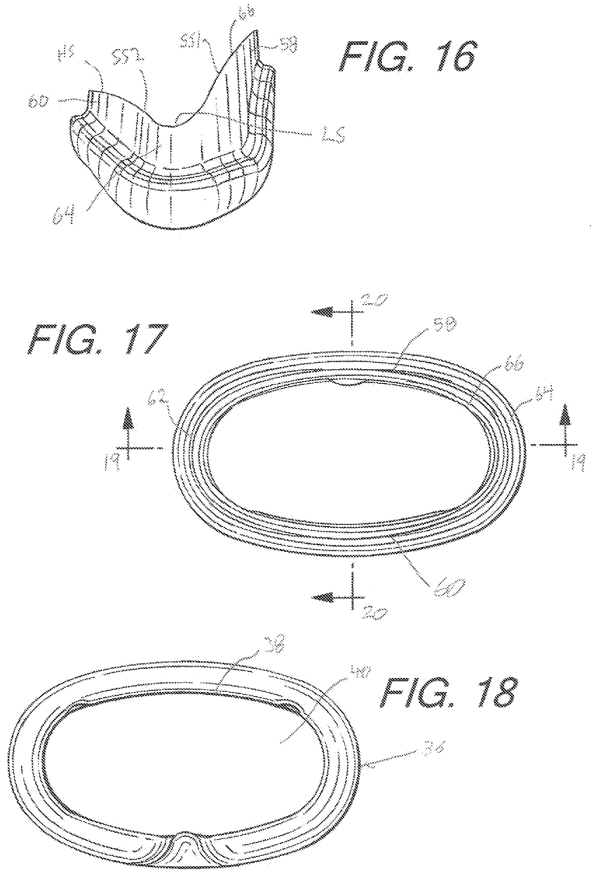

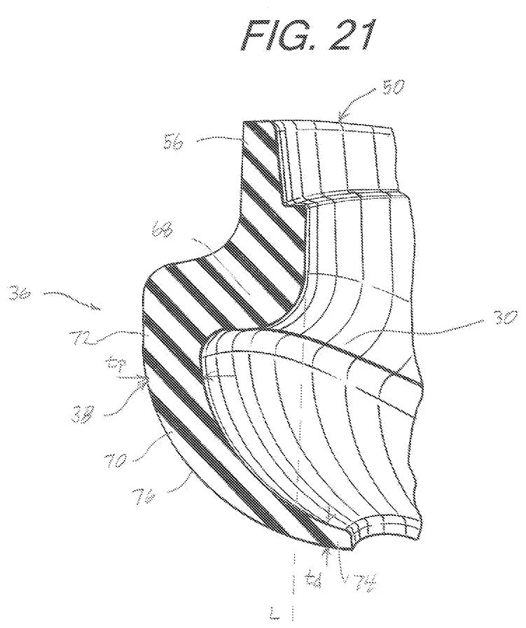

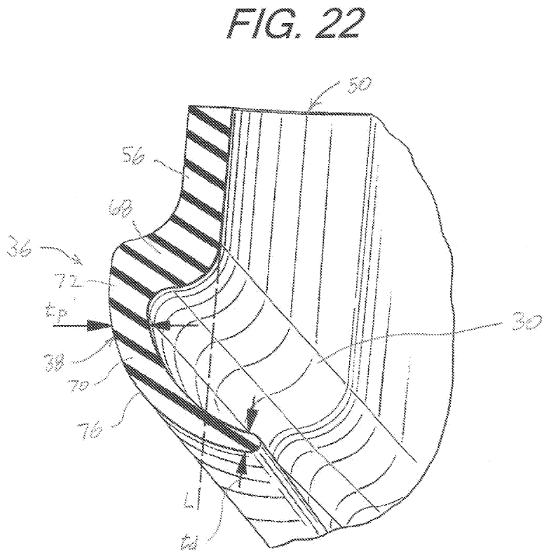

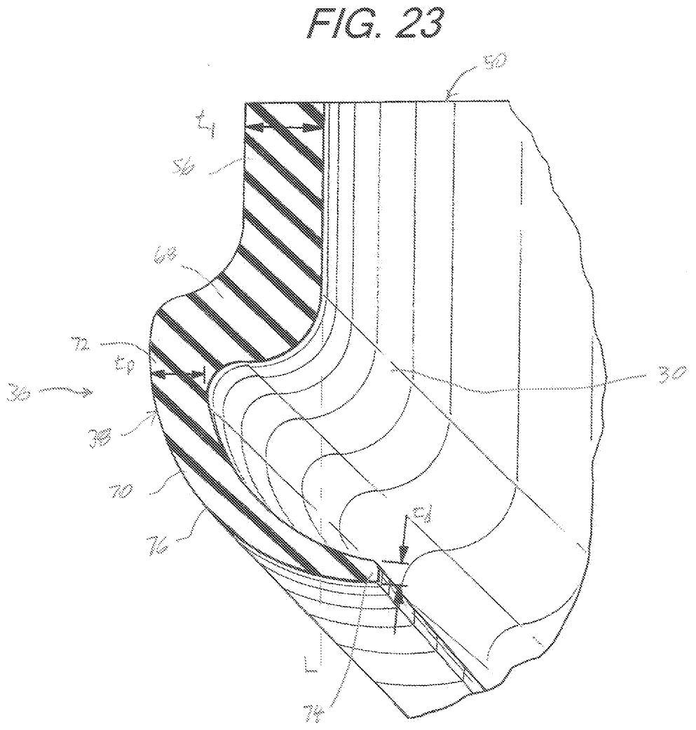

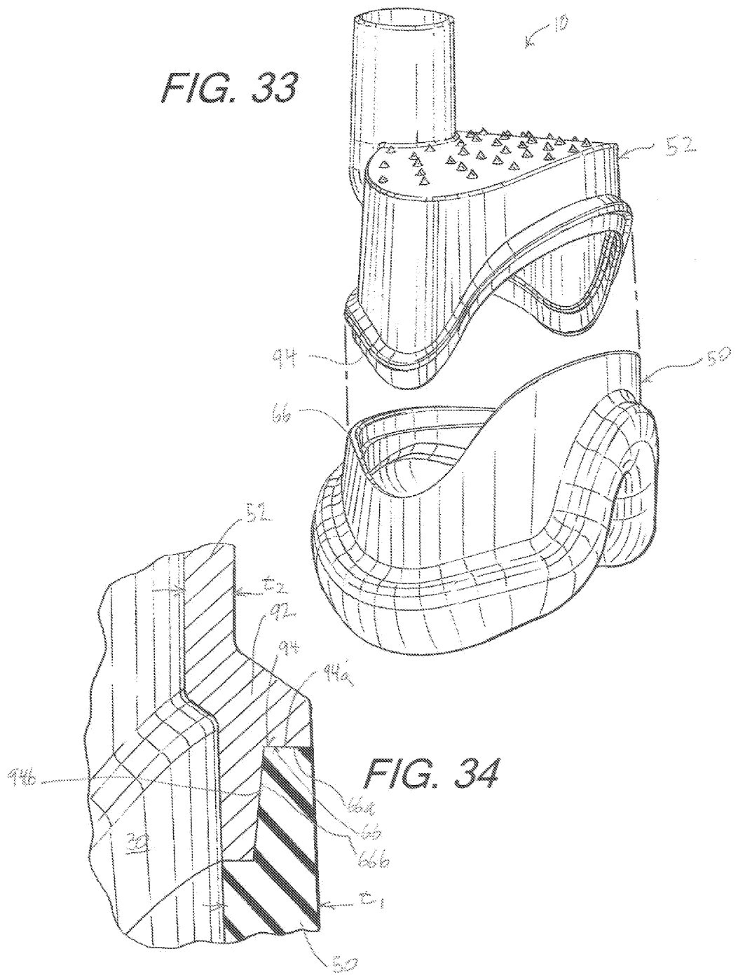

In a first exemplary embodiment, the nasal ventilation mask is shown in FIGS. 3-37 and is designated with the reference numeral 10. The nasal ventilation mask 10 generally includes a housing body 12 and a connector assembly 14. It is understood that in one exemplary embodiment, the nasal ventilation mask 10 can have a single, one-piece integral configuration having a sidewall and other components. In such a configuration, the housing body 12 is integral with the connector assembly 14. In a further exemplary embodiment, the nasal ventilation mask 10 has a multi-component configuration such as shown in FIGS. 3-37 wherein multiple components are operably connected together. General structural configurations of the nasal ventilation mask 10 are described herein, and structures of the multiple components that form the mask 10 and respective connection details are also described herein for the exemplary embodiment of FIGS. 3-37.

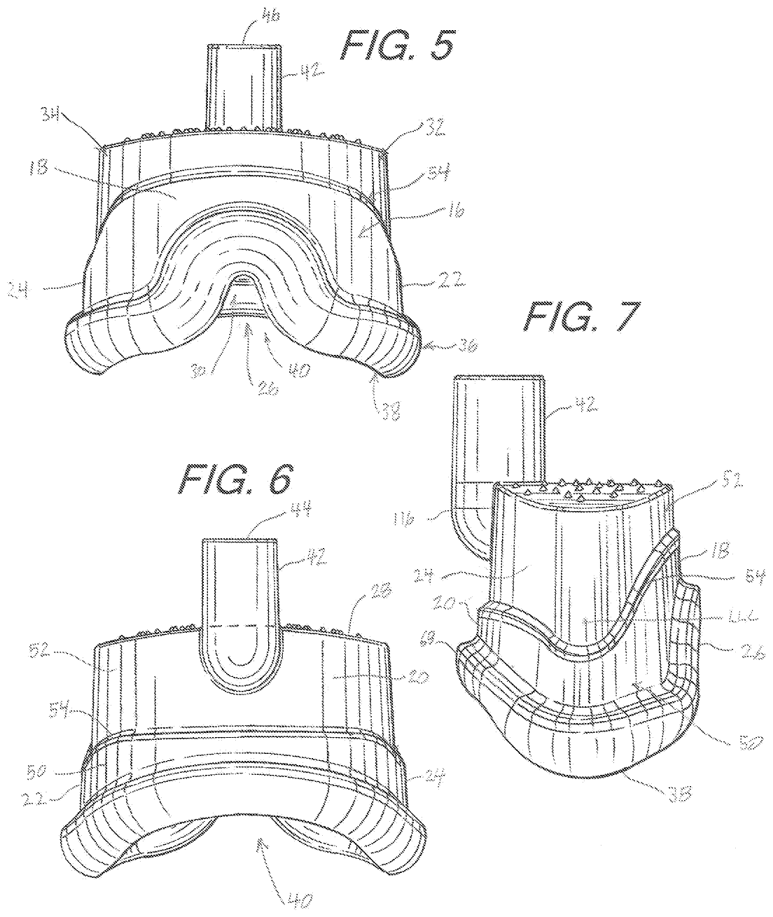

As further shown in FIGS. 3-37, the nasal ventilation mask has a housing body 12 wherein the connector assembly 14 operably extends from the housing body 12. The housing body 12 has a sidewall 16 that extends fully around the housing 12. The sidewall 16 may be referred to as a circumferential sidewall 16 having a contoured configuration. The sidewall 16 may also be referred to as an annular sidewall 16 while not necessarily having a circular cross-section. In this exemplary embodiment, the circumferential sidewall 16 has a generally oval cross-section to be further described below. As further shown in FIGS. 5-11, the sidewall 16 of the housing 12 has a front wall section 18, a rear wall section 20, a first end wall section 22 and a second end wall section 24. In an exemplary embodiment, the rear wall section 20, the first end wall section 22 and the second end wall section 24 are generally plain and uninterrupted and void of any openings therethrough. It is understood that the front wall section 18 is generally opposite the rear wall section 20. The first end wall section 22 is generally opposite to the second end wall section 24. The end wall sections 22,24 extend between the front wall section 18 and the rear wall section 24. As the sidewall 16 has a contoured configuration, it is understood the wall sections 18-24 smoothly transition from one wall section to the next adjacent wall section circumferentially around the mask 10. The front wall section 18 of the sidewall 16 further defines a slot 26 to receive a portion of a nose of a patient to be further described. The slot 26 has a slot height SH also to be further described (FIG. 14) and it is understood the lower end of the slot 26 can be measured at different locations. The housing 12 further has a top wall 28 operably connected to the sidewall 16. In this exemplary embodiment, the top wall 28 is generally transverse to the sidewall 16 although the top wall 28 can have some curvature. The housing 12 defines an internal cavity 30 wherein the sidewall 16 and top wall 28 cooperate to define the internal cavity 30. Proximate generally an interface area between the top wall 28 and the end wall sections 22,24, the housing 12 defines a first shoulder 32 and a second shoulder 34. It is understood the shoulders 32,34 can be positioned on the top wall 28 or also a combination of the top wall 28 and adjacent sections of the sidewall 16. The shoulders 32,34 are generally rigid in construction and are engaged by an emergency responder or medical worker when using the nasal mask 10 to provide an enhanced seal as described in greater detail below.

The housing 12 further has a peripheral end 36 or a distal end 36 wherein the housing 12 has a sealing member 38 proximate the distal end 36. The sealing member 38 has structures for sealing against a face of a patient. The sealing member 38 is an air pressure sealing member so that air pressure in the internal cavity 30 of the mask 10 is not lost through the seal to be described in greater detail below. At the peripheral end 36 or distal end 36, the housing 12 defines a distal end opening 40. The end opening 40 is in fluid communication with the internal cavity 30. It is understood that the end opening 40 is further in fluid communication with the slot 26. In an exemplary embodiment, the slot 26 has a height dimensioned to only extend to a dorsal base of a patient's nose as explained in greater detail below. In other exemplary embodiments, the slot 26 and other portions of the mask 10 can be designed such that the slot 26 can reach the dorsal bridge of the nose. The distal end opening 40 is placed over a nasal airway of a patient and the slot 26 is positioned over the nose to be described in greater detail below. As will be further described below, the sealing member 38 extends around a fully periphery of the end opening 40 and including the slot 26. It can further be appreciated from FIGS. 5-11 that the mask 10 is configured such that the nose slot 26 is positioned generally transverse to the end opening 40 at the peripheral/distal end 36 of the mask 10. It is further understood that the sealing member can be an integral portion of the housing 12 or a separate structural member operably attached to the housing 12.

As further shown in FIGS. 5-11, the connector assembly 14 extends from a proximal end of the housing 12 and is generally located opposite the distal end 36 of the housing 12. In one exemplary embodiment, the connector assembly includes a receiver member 42. The receiver member 42, which can be referred to as a chimney structure, is a generally a slight conical tube member that extends from the top wall 28 of the housing 12. In an exemplary embodiment of the present invention, the receiver member 42 has a portion that also extends from the rear wall of the housing 12 that will be described in greater detail below. The receiver member 42 defines an internal conduit 44 that is in fluid communication with the internal cavity 30. The receiver member 42 further has an inlet opening 46 in fluid communication with the internal conduit 44, and the receiver member 42 further defines an outlet opening 48 at an end of the internal conduit 44 opposite the inlet opening 46 that is in fluid communication with the internal cavity 30. As described in greater detail below, the outlet opening 48 is positioned cooperatively in the top wall 28 and rear wall section 20 in an exemplary embodiment of the invention. The receiver member 42 is dimensioned to be operably connected to an inflatable bag of a manual resuscitator bag assembly to be described in greater detail below. It is understood that there is fluid communication from the inlet opening 46, the internal conduit 44, outlet opening 48, internal cavity 30 and distal end opening 40, thereby defining a passageway for a breathing gas to flow through the mask 10 as further described below. Furthermore, while there is some offset structural configuration to be described below, this passageway has portions that are generally in an inline configuration traveling generally linearly through the mask 10 as can be appreciated from FIG. 11.

As discussed, the nasal ventilation mask 10 of FIGS. 3-37 is formed from multiple components in one exemplary embodiment of the invention. The nasal ventilation mask 10 has a two-piece construction. As further shown in FIGS. 5-11, the nasal mask 10 has a housing body segment 50 and a connector segment 52. The housing body segment 50 is operably connected to the connector segment 52 wherein the housing body 12 and the connector assembly 14 are formed as described above. Once connected as shown for example in FIGS. 3-4, the nasal ventilation mask 10 has a connection joint 54 defined therebetween and generally has the same structures and function as described herein. The connection joint 54 will be described in greater detail below.

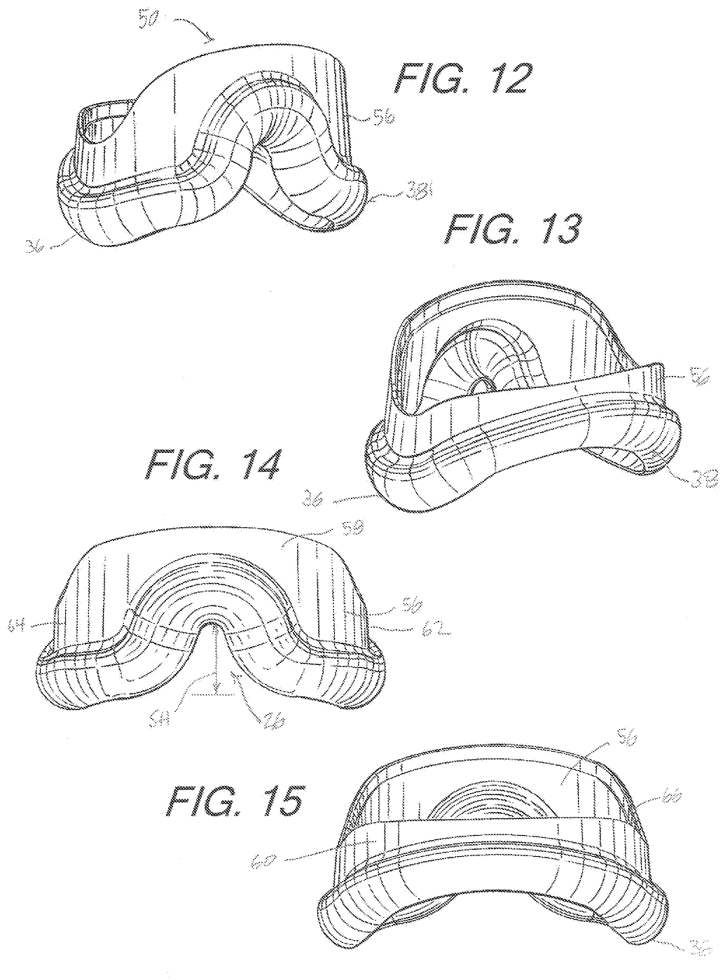

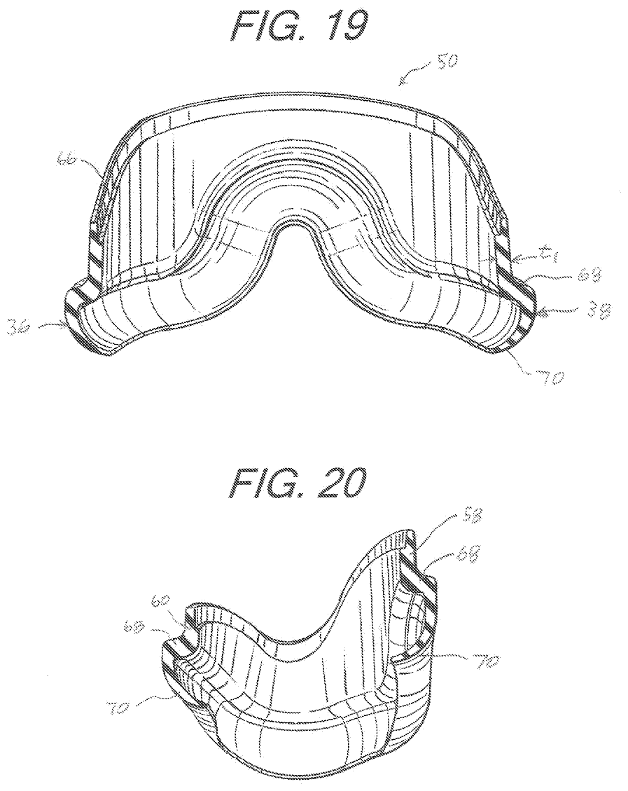

FIGS. 12-23 further show the housing body segment 50. The housing body segment 50 generally forms a lower portion of the nasal ventilation mask 10, and thus defines, among other structures, the distal end 36 of the mask 10 as well as the sealing member 38. The housing body segment 50 has a lower sidewall segment 56 that forms the lower portion of the sidewall 16 of the housing 12. The lower sidewall segment 56 defines an internal area that will cooperate to form the internal cavity 30. The lower sidewall segment 56 also defines a front wall section 58, a rear wall section 60, a first end wall section 62 and a second end wall section 64. It is understood that the wall sections 58-64 cooperate with other wall sections to be described to form the wall sections 18-24 of the mask 10 previously described. The front wall section 58 contains the nose slot 26 wherein the slot 26 is defined completely in the front wall section 58. As further shown, the front wall section 58 has a greater height/length dimension than the rear wall section 60. The front wall section 58 has a convex upper perimeter. The lower sidewall segment 56 has a proximal end that defines a first connection surface 66 and facing upwards and away from the distal end 36. It is understood that the first connection surface 66 could take various configurations such as a combination of planar and angled surfaces. As can be appreciated from FIGS. 12-16, the first connection surface 66 has a greater height from the distal end 36 at the front wall section 58 than a height of the first connection surface 66 from the distal end 36 at the rear wall section 60. In addition, the first connection surface 66 slopes downwardly from the front wall section 58 towards the rear wall section 60 along a curved path. The first connection surface 66 may be considered to slope along generally a conic curve. At proximate a lateral midpoint of the housing body segment 50 between the front wall section 58 and the rear wall section 60, the first connection surface 66 has a lowermost segment LS (FIG. 11). The first connection surface 66 further has a generally horizontal segment HS extending from the rear wall section 60 which then also slopes downwardly to the lowermost segment LS. Thus, as shown in FIG. 16, the first connection surface 66 has a first sloped segment SS1 from the front wall section 58 and a generally horizontal segment HS towards the rear wall section 60 which then extends to a second sloped segment SS2 from the generally horizontal segment HS. As further shown in FIGS. 19, the lower sidewall segment 56 has a first thickness t₁, wherein the first thickness t₁ is 0.070 inches or 0.090 inches in an exemplary embodiment. It is understood that the thickness can be varied. For example, in another exemplary embodiment, the lower sidewall segment 56 could have a thickness of 0.125 inches.

FIGS. 19-23 further show additional structure and detail at the peripheral end 36 or the distal end 36 of the housing body segment 50. At proximate the distal end 36, the housing body segment 50 has a reinforcing member 68 extending from an outer surface of the lower sidewall segment 56. Thus, the reinforcing member 68 is generally normally to the lower sidewall segment 56 and has a cantilevered configuration (FIG. 21). As further shown in FIGS. 21-23, the reinforcing member 68 can have an upper surface having a radius at an interface with the outer surface of the sidewall 16 wherein an angle of 15° is formed as shown. The reinforcing member 68 can still be considered generally or approximately normal or transverse to the sidewall 16 of the housing 12. FIGS. 21-23 further show that in this configuration, the sealing member 38 and lip member to be described, while being connected to the housing 12 proximate the end opening 40, the sealing member 38 is remote from the sidewall 16 via the reinforcing member 68. The reinforcing member 68 may have a thickness of approximately 0.090 inches and extend outwardly from the sidewall 16 approximately 0.10 inches in an exemplary embodiment. In another exemplary embodiment, the reinforcing member 68 may have a thickness of approximately 0.120 inches and extend outwardly from the sidewall 16 approximately 0.125 inches. In a further exemplary embodiment, the reinforcing member 68 is integral with the housing body segment 50. As described in greater detail below, as the housing body segment 50 is formed of a more flexible material, the reinforcing member 68 provides an amount of rigidity in certain areas of the mask 10 to resist buckling as desired. It is understood that FIGS. 21-23 show the housing body segment 50 can have varying thicknesses and connection surfaces 66. As further shown in FIGS. 19-23, the housing body segment 50 has the sealing member 38 at the distal end 36. In particular, the sealing member 38 depends from an outer end of the reinforcing member 68 in an exemplary embodiment. The sealing member 38 has a lip member 70. The lip member 70 has a proximal end 72 integrally connected to the outer end of the reinforcing member 68. The lip member 70 is inwardly curled wherein a distal end 74 of the lip member 70 extends towards the internal cavity 30, and into the internal cavity 30 in an exemplary embodiment. The lip member 70 has a proximal thickness tₚ at the proximal end 72. The lip member 70 is tapered towards the distal end 74 wherein the distal end 74 of the lip member 70 has a distal thickness t_{d}. The distal thickness t_{d} is less than the proximal thickness tₚ. It is understood that the thickness of the lip member 70 gradually decreases towards the distal end 74 of the lip member 70. As further shown in FIG. 21, the housing body segment 50 defines an inner surface (and an inner surface of the sidewall 16) and an imaginary line L extends from the inner surface and beyond the distal end 36. The lip member 70 extends past the imaginary line L wherein the distal end 74 of the lip member 70 extends past the imaginary line L and towards the internal cavity 30. It is understood that the length of the lip member 70 can vary wherein the distal end 74 of the lip member 70 extends just to the imaginary line L or short of the imaginary line L. As described, the lip member 70 depends from an outer end of the reinforcing member 68. In particular, the proximal end 72 depends from the free cantilevered end of the reinforcing member 68. Thus, the proximal end of the lip member 70 is generally offset from the lower sidewall segment 56 as well as the overall sidewall 16 of the mask 10. The proximal end 72 of the lip member 70 is outwardly offset from the lower sidewall segment 56 and further remote from the internal cavity 30. In other exemplary embodiments, the lip member 70 can be aligned with the lower sidewall segment 56. In an exemplary embodiment, the housing body segment 50 or sidewall 16, the reinforcing member 68 and the lip member 70 are all integral with one another. As further shown in FIGS. 21-23, the lip member 70 has an outer surface 76 that engages a face of the patient in use to be described. The outer surface 76 of the lip member 70 has a generally convex shape and is generally smooth and uninterrupted. In one exemplary embodiment, the proximal end thickness tₚ is 0.060 inches or in the range of 0.040-0.080 inches, and the distal end thickness t_{d} is 0.025 inches or in the range of 0.015-0.040 inches. The thicknesses of the lip member 70 can vary depending on various factors such as sealing requirements or the durometer of the material of the lip member 70.

Thus, as can be appreciated from FIGS. 19-23, the reinforcing member 68 is generally incorporated with the sealing member 38 and, in particular, the lip member 70. The reinforcing member 68 extends generally transverse to the lower sidewall segment 56 and sidewall 16 of the mask 10. The reinforcing member 68 is incorporated into, or integrated into an outer end of the sidewall 16. The inwardly curled lip member 70 depends from the reinforcing member 68 and extends into the internal cavity 30. In this configuration, the lip member 70 of the sealing member 38 is connected proximate the end opening of the housing 12 but remote from the sidewall 16 as the lip member 70 is positioned outwardly away from the sidewall 16. Further in this configuration, the reinforcing member 68 and lip member 70 provide an integrated reinforcing structure that minimizes potential floppiness in the mask 10. Such integration provides a cleaner, enhanced design as well for allowing more efficient resin flow in the mold when forming the component. In addition, the depending and cantilevered configuration enhances the seal achieved by the lip member 70 in providing smooth inward deflection of the lip member 70.

The housing body segment 50 can also have the generally oval footprint or configuration generally at the distal end 36 or approximately oval as shown for example in FIGS. 17-18. Thus, the distal end opening 40 may have an oval configuration to be placed on the face of a patient. As discussed in greater detail below, other exemplary embodiments may have more of an obround configuration at the distal end 36.

As discussed, the housing body segment 50 is made from a flexible elastomeric material having a low or soft durometer value. In certain exemplary embodiments, the material can be thermoplastic polyurethane (TPU), thermoplastic elastomer (TPE) or low durometer silicone. In further examples, such materials can have a Shore A durometer in the range of 20-50. In other exemplary embodiments, the Shore A durometer could be as high as 55. In still a further exemplary embodiment, the Shore A durometer can be in the range of 30-40, or 40 Shore A durometer in a particular exemplary embodiment. In an exemplary embodiment, the housing body segment 112 has a part material volume of 1.042 cubic inches.

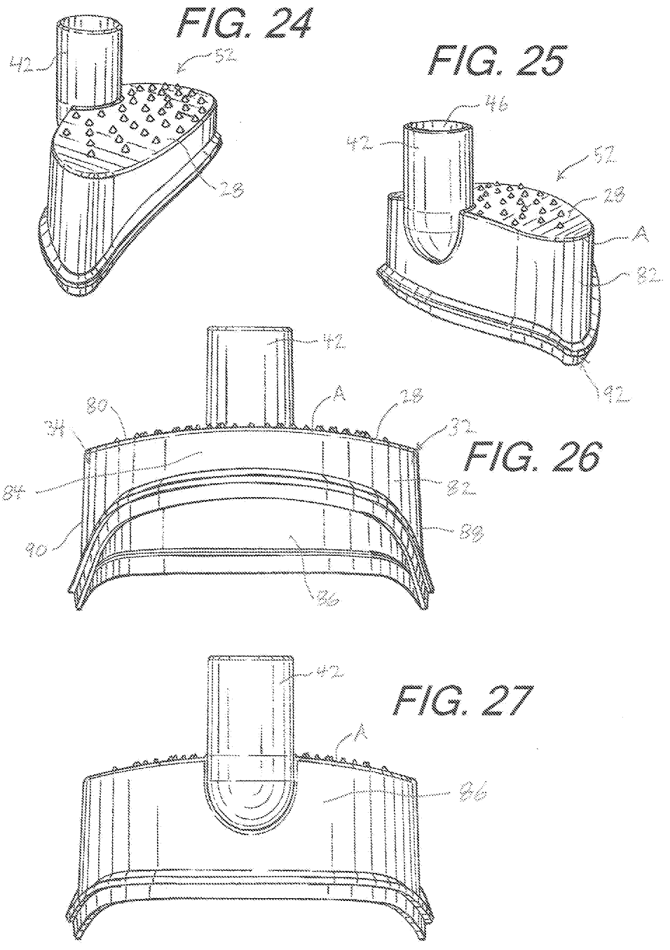

FIGS. 24-30 further disclose the connector segment 52. As discussed, the connector segment 52 cooperates with and is operably connected to the housing body segment 50 to form the nasal mask 10 as described in greater detail below. The connector segment 52 generally forms an upper portion of the nasal mask 10. The connector segment 52 has a base portion 80 that generally corresponds to the top wall 28 of nasal mask 10. The connector segment 52 further has an upper sidewall segment 82 that generally depends down from the base portion 80. The base portion 80, or top wall 28, in cooperation with the upper sidewall segment 82 defines an internal area that cooperates with the internal area defined by the housing body segment 50 to define the internal cavity 30. It is further understood that the base portion 80, or top wall 28 meets with the upper sidewall segment 82 at an interface area A. In other embodiments, the base portion 80, or top wall 28 may be generally recessed from an upper most portion of the upper sidewall segment 82. This configuration assists in providing tactile feedback for emergency responders when pressing on the mask 10. As discussed below, the emergency responder, with fingers or a thumb and finger, may press down on the top wall 28 to assist in generating an airtight seal and the recessed configuration helps better achieve this action, or having other gripping members to be described.

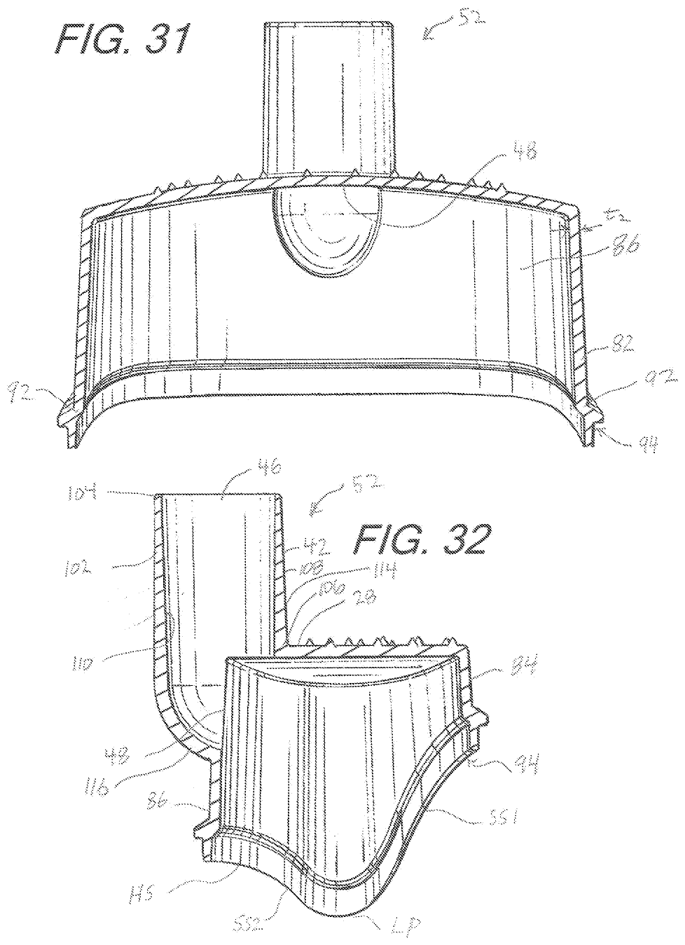

Similar to the lower sidewall segment 56 of the housing body segment 50, the upper sidewall segment 82 defines a front wall section 84, a rear wall section 86, a first end wall section 88 and a second end wall section 90. As further shown, the front wall section 84 has a shorter dimension than the rear wall section 86. The front wall section 84 has a concave lower perimeter. The upper sidewall segment 82 has a proximal end that is integrally connected to the base portion 80 and top wall 28 at the interface area A. These connections further define the first shoulder 32 and the second shoulder 34 at respective interface areas A between the top wall 28 and the upper sidewall segment 82. It is understood that such connections are integral in an exemplary embodiment of the invention. As shown in FIG. 31, an angled transition connector 92 is formed generally at a distal end of the upper sidewall segment 82. The transition connector 92 extends outwardly from the upper sidewall segment 82. Thus, the upper sidewall segment 82 is positioned inward of the transition connector 92 or the upper sidewall segment 82 is inwardly offset from the transition connector 92. Thus, transition connector 92 extends circumferentially inward from the housing body segment 50 wherein the upper sidewall segment 82 is offset circumferentially inward from the lower sidewall segment 56 of the housing body segment 50. In one exemplary embodiment, the angled transition connector 92 is integral with upper sidewall segment 82. A free end of the angled transition connector 92 defines a second connection surface 94 that faces downward away from the top wall 28. It is understood that the second connection surface 94 could take various configurations such as a combination of planar and angled surfaces, but generally has a cooperative configuration to be operably connected to the first connection surface 66. The second connection surface 94 is closer in proximity to the top wall 28 at the front wall section 84 than the second connection surface 94 at the rear wall surface 86. In addition, the second connection surface 94 slopes downwardly from the front wall section 84 towards the rear wall section 86. The second connection surface 94 may be considered to slope along generally a conic curve in one exemplary embodiment. Other sloped curves are also possible with the connector segment 52 as well as the housing body segment 50. As can be appreciated from FIG. 32, at proximate a lateral midpoint of the connector segment 52 between the front wall section 84 and the rear wall section 86, the second connection surface 94 has a lowermost point LP in relation to the top wall 28. The connection surface 94 has a first downwardly sloped segment SS1 from the front wall section 84 towards the rear wall section 86. A generally horizontal segment HS (or a segment that is considered generally tangential to a horizontal plane) extends from the rear wall section 86. A second downwardly sloped segment SS2 extends downwardly from the horizontal segment HS towards the lowermost point LP in relation to the top wall 28. As further shown in FIG. 31, the upper sidewall segment 82 has a second thickness t₂, wherein the second thickness t₂ is 0.045 inches in one exemplary embodiment. It is understood that the thickness can be varied. In addition, the top wall 28 has a thickness of 0.050 inches. It is further understood that the free end of the angled transition connector 92 that defines the second connector surface 94 has a thickness of 0.070 inches, or 0.090 inches which generally corresponds to the thickness t₁ of the lower sidewall segment 56.

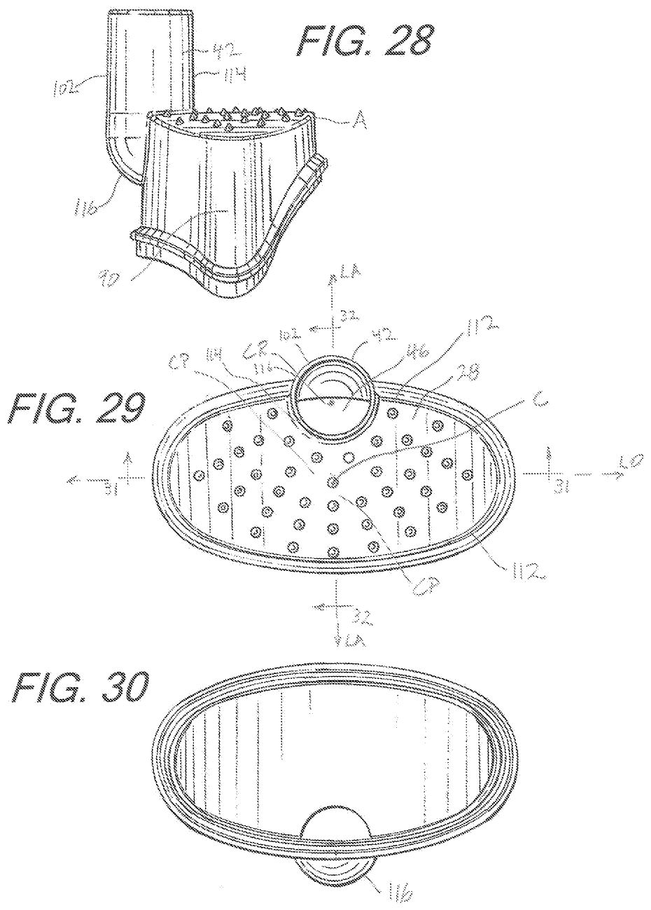

As further shown in FIGS. 28-32, the connector segment 52 supports the receiver member 42, which extends from the top wall 28. As briefly described above, the receiver member 42 defines the inlet opening 46 that is in fluid communication with the internal conduit 44 and outlet opening 48, which is also in fluid communication with the internal cavity 30.

As further shown in FIGS. 27-29 and 31-32, the receiver member 42 extends from the top wall 28 and is designed to be connected to a supply of breathing gas to be discussed in greater detail below. In an exemplary embodiment, the receiver member 42 has a tubular or cylindrical structure thus having a cylindrical wall 102. The receiver member 42 has a distal end 104 that defines the inlet opening 46 and a proximal end 106 defining the outlet opening 48 and further defining the internal conduit 44 therebetween. The outlet opening 48 at the proximal end 106 is generally defined at the housing 12 to be described in further detail below and is in fluid communication with the internal cavity 30 of the housing 12. The inlet opening 46 at the distal end 104 connects to the supply of breathing gas. Additional features of the receiver member 42 are described below.

The receiver member 42 has a tapered outer surface 108, and has a tapered inner surface 110. Thus, the receiver member 42 defines a conical tube. The wall thickness of the receiver member 42 can be in the range of 0.030 inches to 0.045 inches. In certain medical standards for respiratory equipment/conical connectors, a 1.432-degree internal diameter taper is specified. An external approximate 1.5-degree taper (draft angle) on the receiver member 42 is utilized for reliable ejection of the part during the molding process. In one exemplary embodiment, an end forming the inlet opening 46 of the receiver member 42 is formed at approximately 0.030 inches in wall thickness. The wall thickness of the receiver member 42 more proximate the top wall 28 may be 0.074 inches. Thus, the receiver member 42 has a greater wall thickness proximate the top wall 28 and a lesser thickness towards the inlet opening 46. As discussed, the receiver member 42 is dimensioned to operably connect to the inflatable bag of the manual resuscitator assembly. This configuration is dimensioned to conform to International Standard Specifications for conical socket connectors for respiratory equipment. It is understood that the receiver member 42 can have metric dimensions to conform to the International Standard specifications for a 15-millimeter conical socket connector for respiratory equipment. It is further understood that the receiver member 42 can be modified to accept other adapters to, for example, a ventilator, an endotracheal tube or other resuscitator bags.

The connector segment 52 is made from a more rigid material. In exemplary embodiments, the connector segment 52 can be made from acrylonitrile butadiene styrene (ABS) or polycarbonate. As will be discussed in greater detail below, these materials are more rigid than the materials forming the housing body segment 50. Other rigid materials are also possible for the connector segment 52. In a further exemplary embodiment, the connector segment 52 has a part material volume of 0.458 cubic inches.

FIGS. 24-32 further show the receiver member 42 of the connector segment 52 wherein the receiver member 42 extends away from the top wall 28 of the connector segment 52. In prior designs, the receiver member 42 was generally centered on the top wall 28 wherein the inlet opening 46 and outlet opening 48 were generally concentric with the end opening 40 of the housing 12. In this design, the receiver member 42 is positioned in an offset configuration. With such configuration, additional surface area is provided on the top wall 28 for a user to be able to more efficiently press down on the top wall 28 when using the nasal ventilation mask 10 as will be further described below.

As shown in FIG. 29, the top wall 28 defines an outer periphery 112 and a center point C generally at the intersection of a longitudinal axis LO and a lateral axis LA associated with the structure of the top wall 28. Thus, the center point C is positioned within the outer periphery 112. A central portion CP of the top wall 28 generally surrounds proximate the center point C wherein the central portion CP is generally circumjacent the center point C. The central portion CP extends around the center point C but does not extend to the outer periphery 112 of the top wall 28. In an exemplary embodiment, the receiver member 42 is positioned remote from the central portion CP. Further, the receiver member 42 is positioned remoted from the center point C. In particular, the receiver member 42 is positioned proximate the outer periphery 112 of the top wall 28. In such configuration, the inlet opening 46 of the receiver member 42 is considered non-concentric with the end opening 40 of the housing 12. Such positioning provides greater contiguous, uninterrupted surface area on the top wall 28. It is further understood that the receiver member defines a receiver center point CR that is generally at a center of cylindrical wall 102. The receiver center point CR is remote from the center C of the top wall 28 and further remote from the central portion CP. This configuration thus positions the receiver member 42 closer to the rear wall 20 of the housing 12 of the mask 10. In turn, when the mask 10 is positioned on a patient, the receiver member 42 is positioned closer towards the tip of the nose and above the upper lip of the patient. It is further understood that when the mask 10 is positioned on the face of the patient, the receiver member 42 is generally perpendicular to the face of the patient. The receiver member 42 would not extend generally parallel to the face of the patient or extend along a length of the nose of the patient or towards the top of the patient's head. The receiver member 42 extends generally away from the outer surface 76 of the lip member 70 of the sealing member 38 of the mask.

In a further exemplary embodiment, the receiver member 42 is structured such that a portion of the receiver member 42 extends past the outer periphery 112 of the top wall 28. Thus, as shown in FIG. 27-32, a first portion 114 of the receiver member 42 extends from the top wall 28 and a second portion 116 of the receiver member 42 extends from the upper sidewall segment 82 of the connector segment 52. Further, the second portion 116 of the receiver member 42 extends from the rear wall section 60 of the housing 12 opposite the front wall section 58 of the housing 12, or the rear wall section 86 of the connector segment 52 opposite the front wall section 84 of the connector segment 52. Further, as the nose slot 26 is defined in the front wall section 58, the receiver member 42 extends from the rear wall section 60 generally opposite the nose slot 26. As discussed, the receiver member 42 defines the cylindrical wall 102 in an exemplary embodiment. Further in this exemplary embodiment and as shown in FIGS. 28 and 32, the first portion 114 of the cylindrical wall 102 extends from the top wall 28 and the second portion 116 of the cylindrical wall 102 extends from the rear wall section 60 at generally opposite the nose slot 26. Thus, part of the cylindrical wall 102 of the receiver member 42 has offset connections wherein the second portion 116 of the cylindrical wall 102 connecting with the rear wall section 60 extends downwardly past the top wall 28 where the first portion 114 of the cylindrical wall 102 extends from the top wall 28. In this configuration, the outlet opening 48 of the receiver member 42 has one segment defined in the top wall 28 and another segment defined in the rear wall section 60 of the housing 12 or upper sidewall section 82 of the connector segment 52 as can be appreciated from FIG. 32. The two segments cooperate to form the outlet opening 48 providing fluid communication between the inlet opening 46 and internal conduit 44 and the internal cavity 30.

As discussed, the receiver member 42 has the second portion 116 of the cylindrical sidewall 102 that extends from the rear wall section 60 of the housing 12. As discussed above regarding the sealing member 38, the housing 12 further has the reinforcing member 68 that also extends from the housing 12. The second portion of the cylindrical wall 102 extending from the rear wall section 60 extends a distance such that the second portion 116 of the cylindrical wall 102 is generally aligned with a peripheral end of the reinforcing member 68 (FIG. 7).

It is further understood that the connector segment 52 is an integral structure in an exemplary embodiment. Thus, the receiver member 42 is integral with the top wall 28 and rear wall section 86 of the connector segment or rear wall section 20 of the housing 12. Other separate connection structures could also be utilized if desired in other alternative embodiments.

It is understood that the receiver member 42 could extend from the housing 12 in other configurations in other exemplary embodiments. For example, the receiver member 42 could extend entirely from the rear wall section if desired.

As can be appreciated from FIGS. 3-7 and 33-34, the housing body segment 50 is operably connected to the connector segment 52 to form the nasal ventilation mask 10. When operably connected, the first connection surface 66 and the second connection surface 94 abut and are joined together to form the connection joint 54. The upwardly facing first connection surface 66 of the housing body segment 50 confronts the downward facing second connection surface 92 of the connector segment 52. In an exemplary embodiment, the connection joint 54 is formed via an over-molding injection process wherein the segments 50,52 are joined together. For example, those skilled in the art understand that the connector segment 52 can be injection molded in a first mold assembly initially and after completion, placed into another mold assembly. The housing body segment 50 can then be injected molded, or over-molded onto the connector segment 52 resulting in the configuration described above and wherein a chemical bond is formed at the connection joint 54 (FIG. 34). In addition, more complex mold assemblies can be used wherein the housing body segment 50 is molded sequentially during the same overall process as the molding of the connector body segment 52. Other connection methods are also possible such as adhesive bonding or other chemical bonding. Interlocking mechanical connections can also be utilized. Combinations of the above-described connection methods can also be utilized. It is further understood that in a two-shot molding process or over-molding process in forming the housing body segment 50 and connector segment 52, the chemical bond is formed between the segments 50,52. The chemical bond is formed between the materials of the two segments 50,52 to form the connection joint 54.

FIGS. 3, 4 and 33-34 further show the connection joint 54 formed between the housing body segment 50 and the connector segment 52. The connection joint 54 generally has a contoured path around the circumferential sidewall 16. It is understood that the connection joint 54 travels around the full periphery or circumference of the mask 10. The contoured path provides enhanced operability of the mask 10. The connection joint 54 is more proximate to the top wall 28 at the front wall section 18 of the mask 10 than at the rear wall section 20 of the mask 10. As shown in FIG. 5, the connection joint 54 has a generally concave configuration. As shown in FIG. 6, the connection joint 54 has a generally horizontal configuration across the rear wall section 20. As previously discussed, the connection joint 54 has a profile in a curved-slanted configuration, or downwardly sloped along a curved path, generally at the end wall sections 22,24 of the mask 10. In particular and as shown in FIGS. 7 and 11, the connection joint 54 generally slopes downwardly in a curved configuration at the first end wall section 22 and the second end wall section 24 from the front wall section 18 towards the rear wall section 20. The connection joint 54 thus has a first curved segment SS1 and a substantially horizontal segment HS more proximate the rear wall section 20 of the mask 10. The substantially horizontal segment HS meets with a second curved segment SS2 that slopes downwardly to a lowermost segment LS. It is understood that the first curved segment SS1 slopes downwardly and the second curved segment SS2 slopes downwardly and generally meet at the lowermost segment LS. The contoured or curved segment of the connection joint 54 can also take other curved forms. In one exemplary embodiment, the connection joint 54 may slope downwardly along generally a conic curve. The connection joint 54 may also be contoured along generally a radial curve or have a configuration of an arc of a circle.

With the curved/slanted configuration of the connection joint 54, it is understood that the housing body segment 50 and the connector segment 52 also possess such curved/slanted configurations as described above. With such configuration, at proximate a lateral/longitudinal central portion LLC at the first end wall section 22 and the second end wall section 24 of the mask 10, the mask 10 is formed of the more rigid material of the connector segment 52. This can be appreciated from FIG. 7. With the curved profile, the more flexible housing body segment 50 is positioned below the lateral/longitudinal central location LLC while the connector segment 52 having more rigid material occupies the lateral/longitudinal central location LLC. With the sloped or contoured connection joint 54 along a downwardly sloped curve (sloping downwardly along a curved path) at the first end wall section 22 and the second end wall section 24, the mask 10 is formed of the more rigid material of the connector segment 52 at a larger area of the end wall sections 22,24 of the mask 10. This configuration assists in further stabilizing the mask 10 and resists undue flaring or bulging when pressing the mask 10 against a patient's skin during use to be described further. Such flaring has an adverse impact in creating an airtight seal against a patient's face. Thus, the configuration provides for more controlled flexibility of the mask 10 at the end wall sections 22,24 to accommodate varying patient facial contours while still providing for an enhanced seal against the face of the patient. Such configuration further provides material cost-saving benefits. In an exemplary embodiment, the curved slanted profile of the connection joint 54 is the result of a conic profile that travels linearly from one side of the mask body to the other side of the mask body in a path generally normal to a central plane of symmetry of the mask body. As discussed, it is understood that the housing body segment 50 and the connector segment 52 have the corresponding and confronting curved slanted profiles.

FIG. 34 discloses the connection joint 54 in greater detail. As discussed, the housing body segment 50 has the first connection surface 66. The first connection surface 66 further defines an upward facing connection surface 66a and an inclined connection surface 66b. The inclined connection surface 66b is tapered generally towards the inner area cooperatively defining the internal cavity 30 of the mask 10. The connector segment 52 has the second connection surface 94. The second connection surface 94 further defines a downward facing connection surface 94a and an inclined connection surface 94b. As described above, the housing body segment 50 is operably connected to the connector segment 52 to form the nasal ventilation mask 10. The respective connection surfaces 66,94 form the connection joint 54. The upwardly facing connection surface 66a of the housing body segment 50 confronts in surface-to-surface engagement the downward facing connection surface 94a of the connector segment 52. The inclined connection surface 66b of the housing body segment 50 confronts in surface-to-surface engagement the inclined connection surface 94b of the connector segment 52. As described above, the connection joint 54 is formed via an over-molding injection process wherein the housing body segment 50 may be over-molded onto the connector segment 52. The connector segment 52 can be molded initially and placed into another mold assembly. The housing body segment 50 can then be injected molded, or over-molded onto the connection segment 52 resulting in the configuration described above and shown in FIG. 34, and wherein a chemical bond is formed at the connection joint 54.

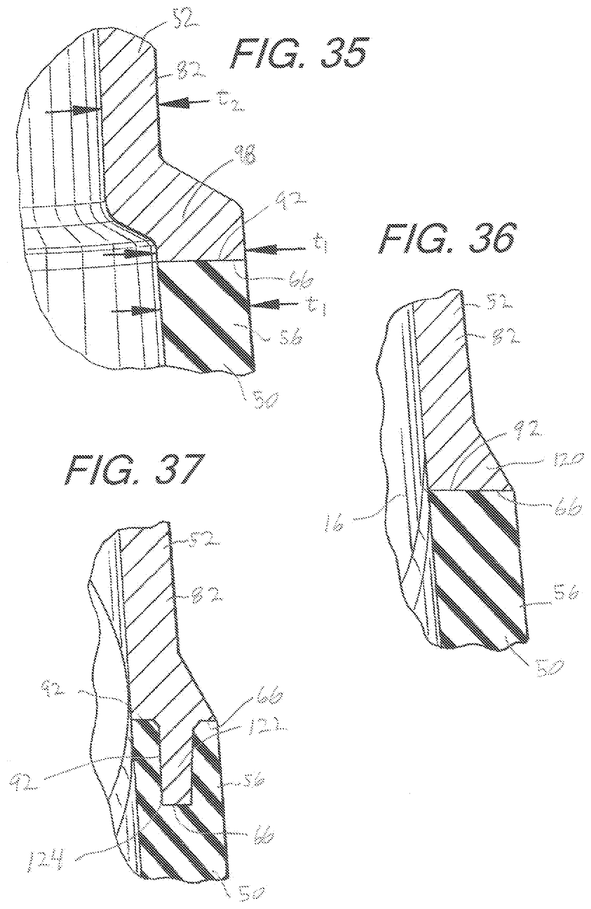

FIGS. 35-37 disclose alternative connection joint configurations. FIG. 35 discloses one alternative configuration of the connection joint 54 of the mask 10. In this embodiment, a butt joint is formed as the connection joint 54. The first connection surface 66 of the housing body segment 50 confronts and abuts in surface-to-surface engagement the second connection surface 94 of the connector segment. The over-molding process described above can also be used in the formation of the connection joint 54.

FIG. 36 further discloses another configuration of the connection joint 54 of the mask 10. The housing body segment 50 has the first connection surface 66 and the connector segment 52 has the second connection surface 94 wherein the respective connection surfaces 66,94 confront one another. In an over-molding process in one exemplary embodiment of the invention, the respective materials of the housing body segment 50 and the connector segment 52 are chemically-bonded in a butt joint to form the connection joint 54. As further shown in FIG. 36, the connector segment 52 has a flared portion 120 that gradually increases the thickness of the sidewall 82 of the connector segment 52 to correspond to the thickness of the sidewall 56 of the housing body segment 50. In an exemplary embodiment, the flared portion 110 forms a transitional wall thickness from 0.045 inches to 0.075 inches. The thickness of the sidewall 56 of the housing body segment 50 is 0.075 inches. It is further understood that the wall thicknesses could be in the range of 0.070 inches to 0.080 inches or 0.090 inches in other exemplary embodiments. The flared portion 120 is dimensioned to have an outwardly flared portion to correspond to the thickness of the lower sidewall segment 56.

FIG. 37 discloses another alternative connection joint 54. The connector segment 52 has a tongue 122 and the housing body segment 50 has a groove 124. The tongue 122 is received in the groove 124. In the over-molding process as described, the segments 50,52 are chemically-bonded to form the connection joint 54. In this tongue/groove configuration, the connection joint 54 has increased bonding surface areas. The tongue/groove configuration provides additional mechanical integrity. It is further understood that one of the housing body segment 50 and the connector segment 52 can have the tongue 122 and the other of the housing body segment 50 and the connector segment 52 can have the groove 124. While a chemical bond is formed in any of the connection joints 54 in an exemplary embodiment, it is understood that other bonding structures can be utilized including adhesive bonding or other fused structures. It is understood that any of the various structures of the connection joint 54 can be used in the various exemplary embodiments of the nasal ventilation mask 10 described herein.

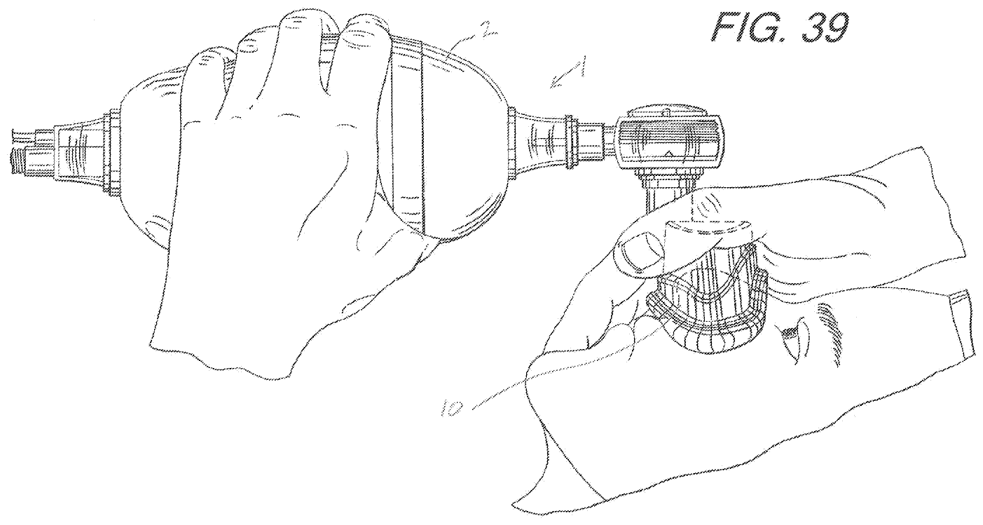

With the nasal ventilation mask 10 fully formed, the mask 10 can be incorporated with an inflatable bag of a manual resuscitator bag assembly. The mask 10 is operably connected to the bag assembly where emergency responders or resuscitators can use the bag assembly on a patient to supply breathing gas to the patient. It is understood that associated fittings may be incorporated into the operable connection between the inflatable bag 2 and the nasal ventilation mask 10. FIG. 39 shows the nasal mask 10 connected to an inflatable bag 2 of a manual resuscitator bag assembly 1. It is understood that the bag assembly 1 has associated fittings to operably connect to the receiver member 42 in accordance with International Standard specifications for connectors for respiratory equipment as discussed herein. Upon such connection, the inflatable bag 2 along with any additional breathing gas supply is in fluid communication with the nasal ventilation mask 10 through the inlet opening 46, internal conduit 44, outlet opening 46 and into the internal cavity 30.

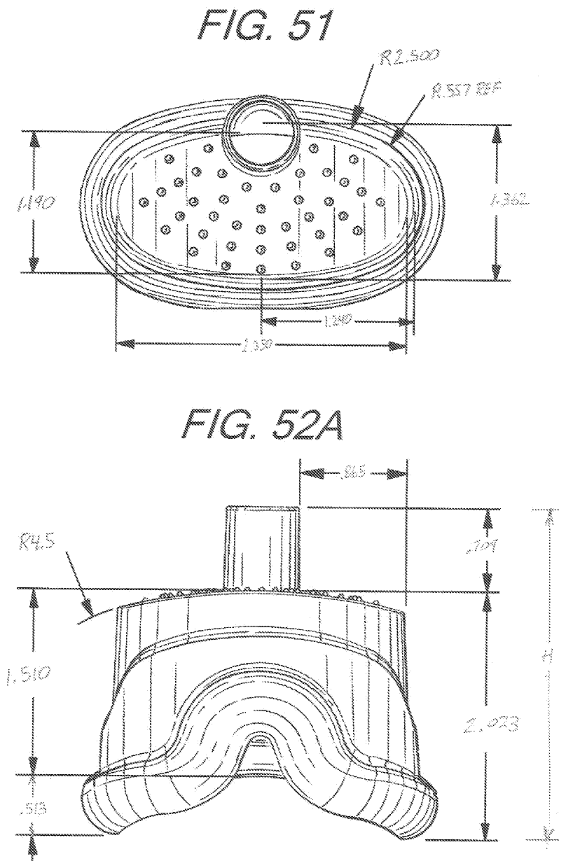

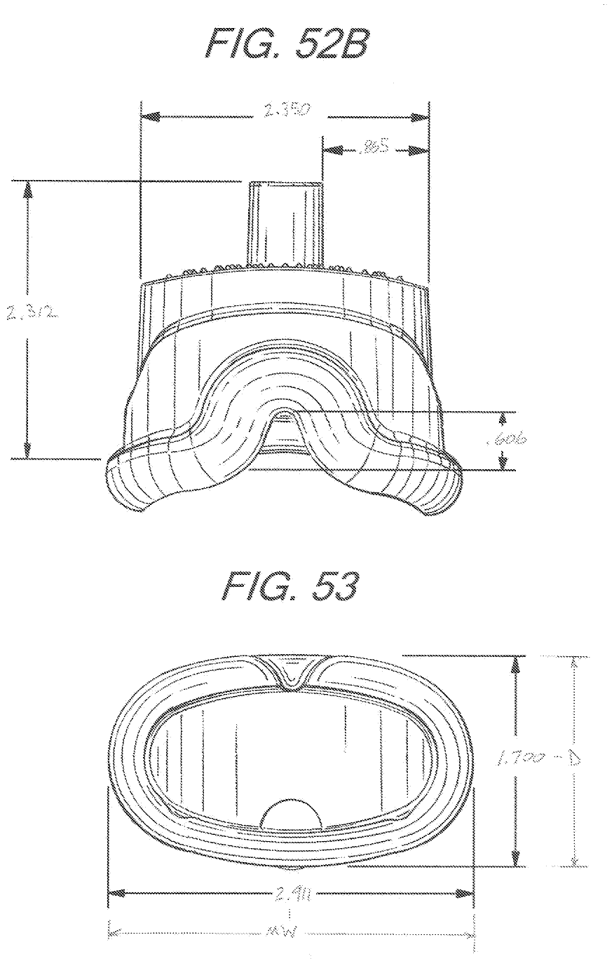

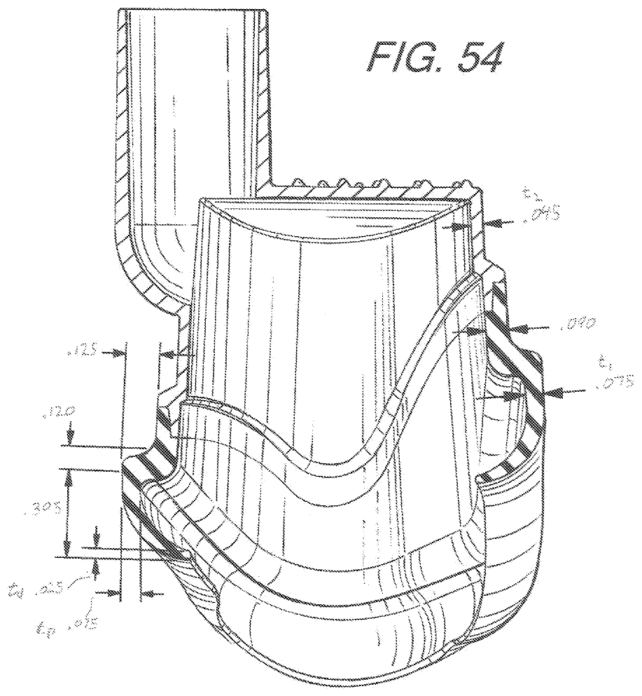

FIGS. 51-54 disclose additional views of the nasal ventilation mask 10 having associated dimensional characteristics thereon in inches. The dimensional characteristics are associated with an exemplary embodiment of the mask 10 of the present invention. It is understood that these dimensions can vary with other exemplary embodiments of the invention or be combined with other particular dimensions of other portions of the mask 10. For example, it is understood that nasal masks 10 of different sizes can be formed to accommodate patients from different ages including infants, small children and adults. In particular, FIG. 51 shows a top plan view of the mask 10 with certain dimensional characteristics. FIGS. 52A-52B show front elevation view with various dimensional characteristics including certain heights (H) and widths and top wall curvatures. FIG. 53 shows a bottom plan view showing width (MW) and depth (D) dimensions of the mask 10. Finally, FIG. 54 shows a cross-sectional view showing certain dimensions including various thicknesses associated with the mask 10. Thus, in the particular exemplary embodiment disclosed, the depth D to height H ration may be in the range of 60%-65% or approximately 62%. The depth D to mask width MW may be in the range of 55%-60% or approximately 58%. Finally, a height H to mask width MW ratio may be in the range of 90%-96% or approximately 94%. It is understood that the dimensions shown are associated with certain exemplary embodiments of the mask 10, and it is understood that the dimensions can include ranges around the dimensions such as +/−30% in other exemplary embodiments. The dimensions of the mask 10 can vary as the mask 10 can be constructed to be used on patients of varying ages, which necessarily results in patient face structures having different sizes such as an infant, child, young adult or grown adult.

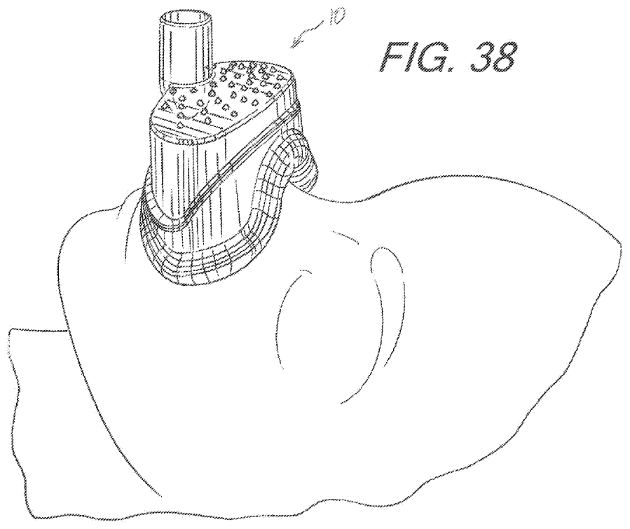

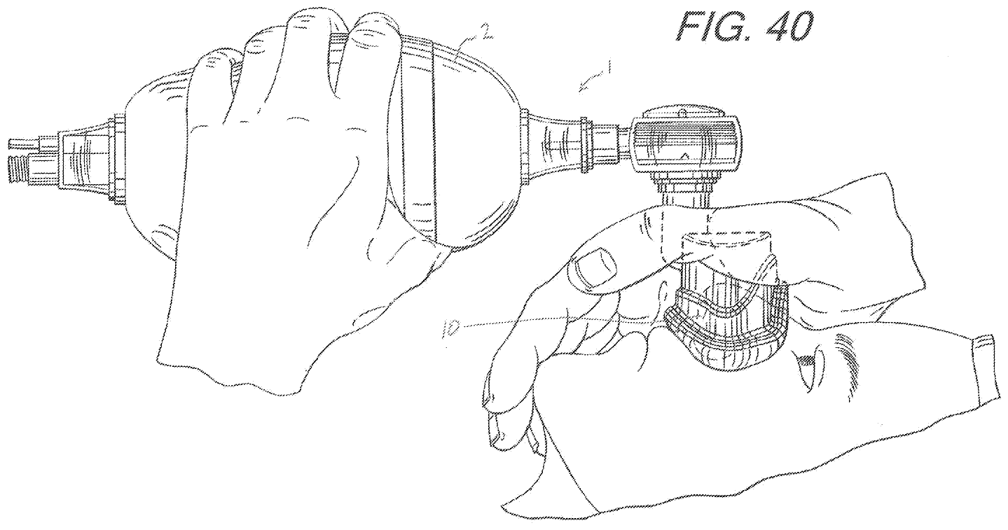

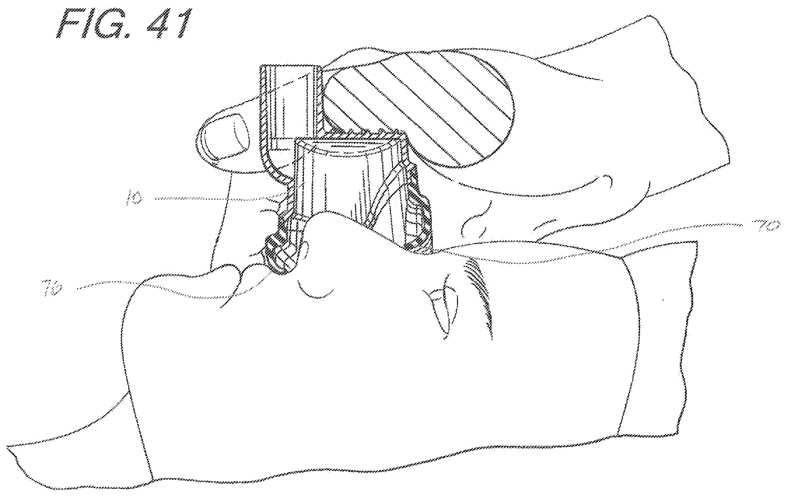

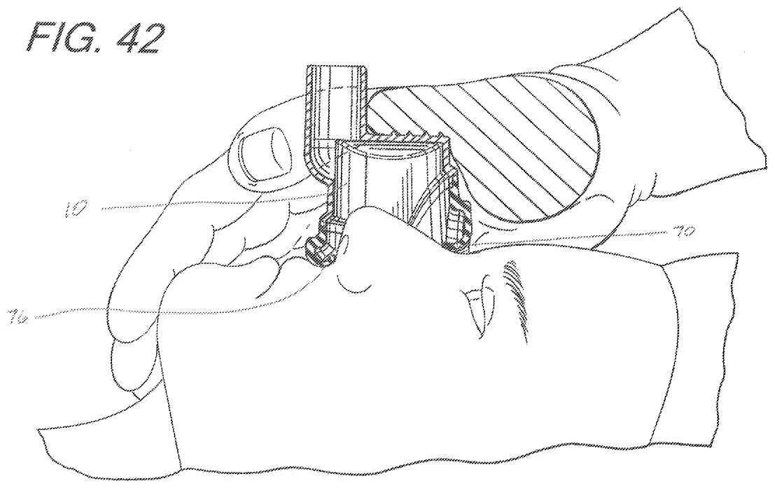

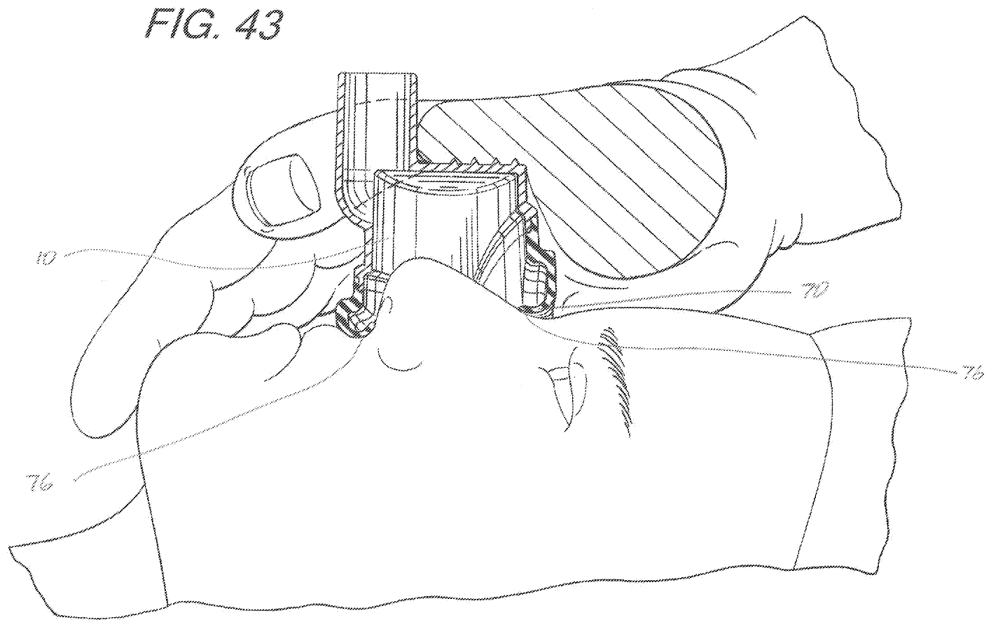

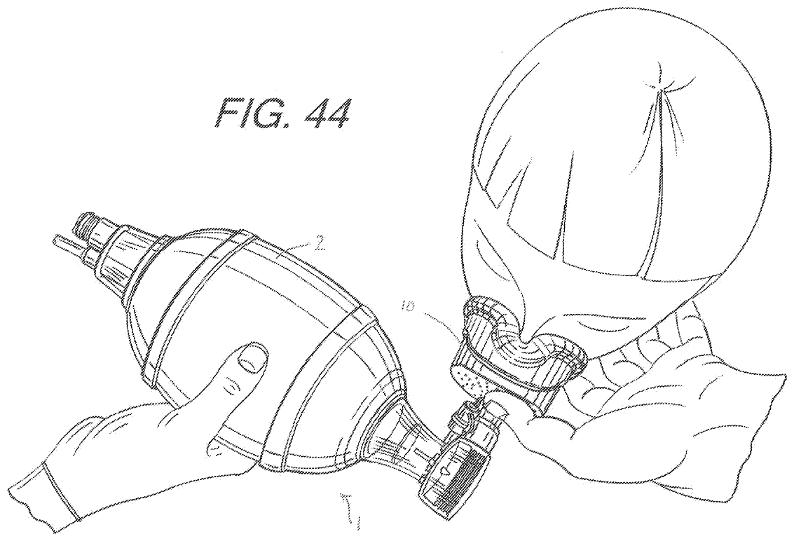

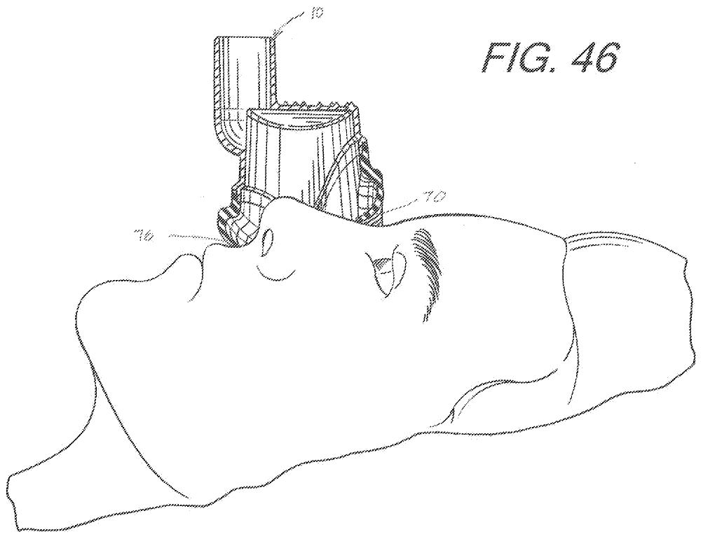

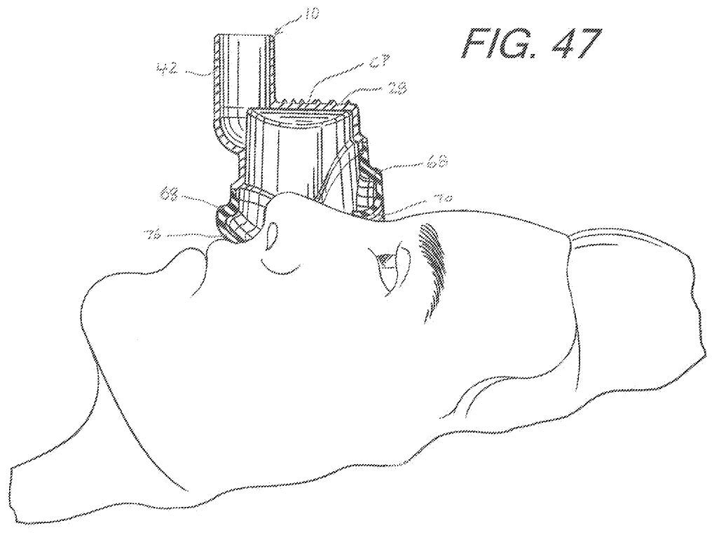

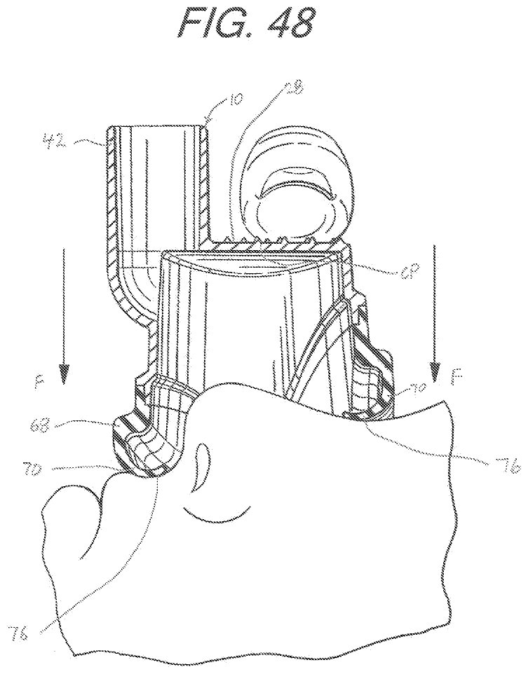

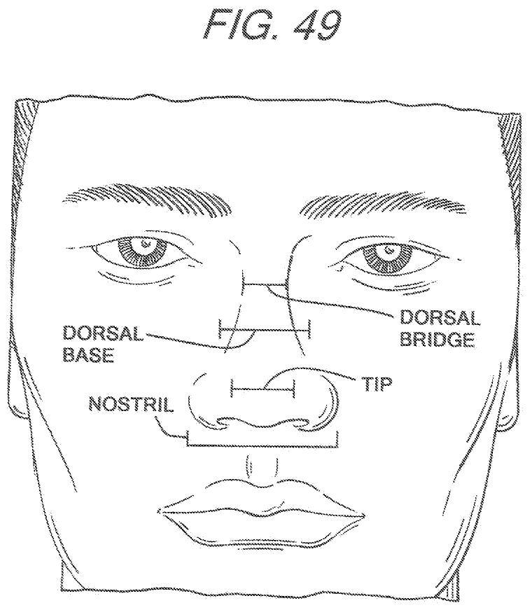

A patient may experience an event requiring breathing assistance. In operation, the nasal ventilation mask 10 (understood already operably connected to the inflatable bag 2 of the bag assembly 1 or oxygen source) is placed over a nose of the patient, e.g., over a nasal airway of the patient. Thus, the nose of the patient is received through the distal end opening 40. The slot 26 also receives a portion of the nose wherein the mask 10 at the slot 26 engages the patient generally at the dorsal base of the nose of the patient. As can be appreciated from FIG. 49, the generally oval footprint of the mask 10 is dimensioned such that the mask 10 at the top of the slot 26 engages the nose at the dorsal base and not at the dorsal bridge of the nose. Engaging at the dorsal base of the nose allows for creating a more consistent airtight seal against the patient's face. It is understood that the mask 10 could be designed wherein the end opening 40 is structured to not need the slot 26. In an exemplary embodiment, however, the slot 26 is in fluid communication with the end opening 40. As described above, the slot 26 is positioned generally transverse to the end opening 40 and rests upon the nose of the patient. As previously described, the receiver member 42 extends generally perpendicular from the top wall 28. When the mask 10 is positioned on the face of the patient as described and shown, the receiver member 42 is generally perpendicular to the face of the patient. The sealing member 38 is engaged with and pressed against the face of the patient to create an airtight seal between the mask 10 and the patient's skin. As can be appreciated in FIGS. 38-47, the sealing member 38 at the distal end 36 of the housing body 12 engages under the nostrils of the nose and above the upper lip of the patient (towards the mouth of the patient). Further portions of the sealing member 38 engage at respective sides of the nose. Finally, portions of the sealing member 38 at the slot 26 engages up the nose, away from the tip of the nose and proximate the dorsal base of the nose as the slot 26 is transverse to the opening 40. The mask 10 is designed and dimensioned with the substantially oval footprint and size (slot height SH) of slot 26 to engage at the dorsal base and not to extend all the way to the dorsal bridge of the nose. Thus, as the mask 10 has the oval configuration and slot 26, the mask 10 engages away from the dorsal bridge and not closer to the patient's eyes. As shown in FIG. 41, the outer surface 76 of the inwardly curled lip 70 of the second sealing segment 30 initially engages the skin of the face of the patient. It is understood that this engagement is fully around the entire end opening 40 including the slot 26. FIG. 46 further shows such configuration. It is understood the lip member 70 may deflect a certain amount in response to the initial placement of the mask 10 on the face of the patient. FIGS. 42-43, shows the mask 10 being further pressed against the face of the patient such as by an emergency responder. FIGS. 47-48 further show such configuration. As shown in FIGS. 39-40, the emergency responder may use a portion of responder's palm, or fingers or a finger and thumb to press against the top wall 28 and/or the shoulders 32,34 of the mask 10 to apply force F to create further sealing. The lip member 70 further deflects proximate the distal end 74 of the lip member 70 wherein the lip member 70 further deflects towards the internal cavity 30 wherein the outer surface 76 further engages the face of the patient. This deflected configuration of the lip member 70 achieves a tight enhanced pneumatic seal and airtight seal between the mask 10 and the patient's skin. The integrated reinforcing member 68 assists in providing stability to further enhance the airtight seal. It is understood that the airtight seal is achieved around the full end opening 40 and including the slot 26. The seal achieved thus maintains air pressure in the internal cavity 30 of the mask.

As further can be appreciated from FIGS. 38-43 and 46-48, the emergency responder can apply force F on the mask towards the patient's face to further enhance the seal. In certain exemplary embodiments, the emergency responder can use, for example, a thumb, or a thumb and finger, or other fingers, to press on the top wall 28 and/or first shoulder 32 and the second shoulder 34. In a further exemplary embodiment, the responder can use a portion of a palm of the hand of the responder to apply force F. In particular, a general thenar region of the hand is positioned proximate the central portion CP of the top wall 28 wherein the receiver member 42 is received between the thumb and forefinger of the responder. The offset configuration of the receiver member 42 provides enhanced ergonomic positioning of the hand of the responder to apply force F proximate the central portion CP of the top wall 28 of the mask 10. As the top wall 28 including the shoulders 32,34 provide a planar, platform-type surface that is rigid, the medical worker can easily apply any requisite force F to the mask 10 towards the patient's face to further enhance the seal (FIGS. 43 and 48). As described above, the receiver member 42 has the off-set configuration wherein the receiver member 42 extends beyond the outer periphery 112 of the top wall 28 and outward from the rear wall section 20 of the housing 12. Thus, when the mask 10 is properly positioned on the patient, the receiver member 42 is positioned closer to the upper lip of the mouth of the patient. The configuration thus provides more specific surface area on the top wall 28 more towards the rear wall section 20. The configuration further provides more contiguous, uninterrupted surface area proximate the central portion CP for the responder to engage. The responder can then apply pressure more efficiently at this specific surface area so that force F can be more efficiently applied more proximate the upper lip of the mouth of the patient which allows the responder to achieve a better seal directly underneath the nose of the patient. This could not be achieved when the receiver member 42 is generally centered on the top wall 28. As further shown in FIGS. 39-40, this configuration thus allows the responder's thumb/finger to be closer towards the mouth of the patient, which further allows for other responder fingers to reach under and cradle a jawline of the patient to achieve additional support when applying force to further enhance the sealing capability of the mask 10. This is particularly beneficial for responders having smaller-sized hands. Further in this configuration, the top wall 28 has more contiguous, uninterrupted surface area capable of being engaged by the responder for applying force F on the mask 10 to create a good seal. In prior designs, the receiver member 42 is centrally-located which interrupted some surface area on the top wall 28. In addition, the structural configuration of the mask 10 allows the responder to apply sufficient force F with a single hand rather than requiring two hands to achieve a sufficient seal as in other prior art designs.

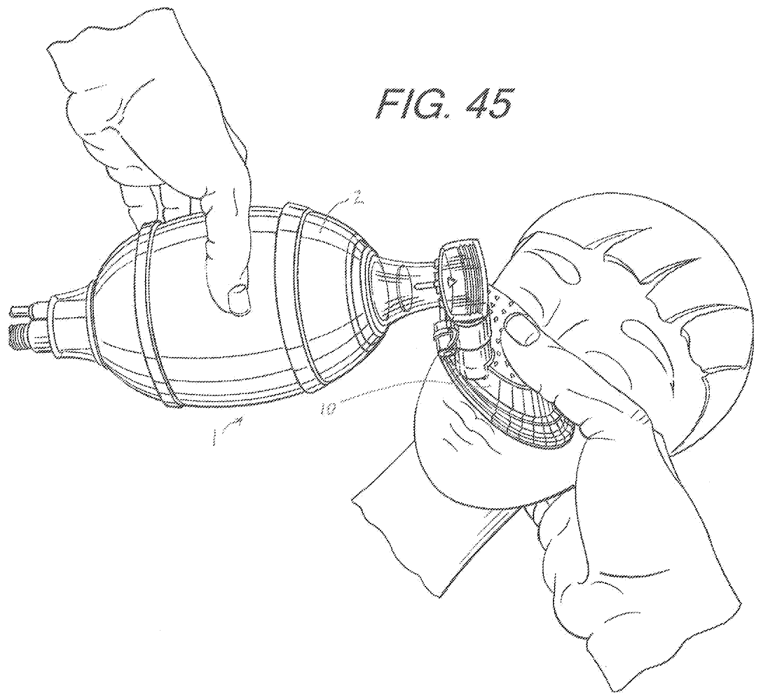

FIGS. 44-45 show additional views of a responder using the mask 10 on a patient. In these views, the responder may press down on the mask 10 in an alternative configuration. As discussed regarding FIGS. 39-40, the responder may be positioned adjacent to and above the patient's head. In FIGS. 44-45, the responder may be positioned adjacent and to one side of the patient. The responder is still in a position to apply force F to the mask 10 and press down on the mask 10 at the central portion CP wherein the outer surface 76 of the lip member 70 engages the patient's skin to provide a seal. The responder may apply such force F with a thumb wherein other fingers can also reach under the patient's jawline to provide additional support when pressing down on the mask 10.

The profile of the mask 10 provides an easy engagement by the emergency responder. Because the nasal ventilation mask 10 only covers the nose, emergency responders or other medical workers have access to the patient's mouth to clean oral passageways such as if the patient vomits or if medical workers need to insert additional medical devices. After treatment on the patient is complete, the nasal mask 10 can be removed from the air bag by the emergency responder or other medical worker.

The nasal ventilation mask 10 can be used with a manual resuscitator bag assembly 1 as described but could also be used with other components or for other applications requiring breathing assistance. For example, the nasal ventilation mask can be used with a ventilator or a continuous positive airway pressure machine. Other components can also be operably associated or integrated with the mask such as oxygen monitors or carbon dioxide monitors or other sensors. Depending on desired configurations, the nasal mask 10 may incorporate additional port structures, adapters or other fittings for operable connection to such components. Such operable integrations with other components can be helpful to medical personnel. For example, integration of a carbon dioxide monitor or sensor assists in determining proper ventilation of the patient. Wireless technology can also be operably associated with the nasal mask 10 and such operable connections.

FIGS. 61-64 disclose additional features that can be incorporated in or operably associated with the mask 10 of FIGS. 3-37. As shown in FIG. 61, the nasal ventilation mask 10 can utilize an adapter member 150. The adapter member 150 is generally a tubular member having a mouthpiece 152 at a distal end. The adapter member is 150 structured to operably attach to the receiver member to be in fluid communication with the internal cavity 30. The responder can use the mouthpiece 152 to blow air directly into the mask 10 in order to inflate the lungs of the patient when circumstances warrant such action. It is understood that the adapter member 150 may include internal valving and other structures to provide and allow for such operation.

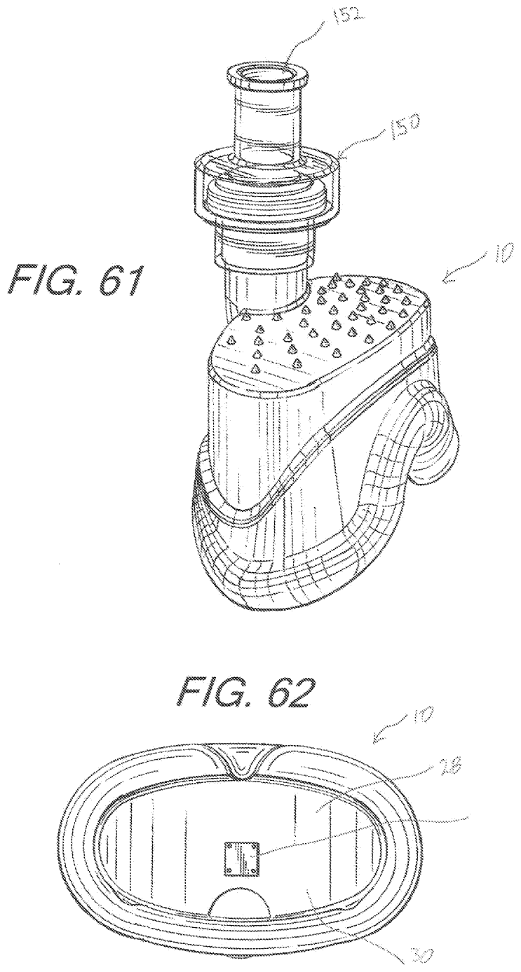

FIG. 62 shows another alternative embodiment of the mask 10 utilizing additional technology in the form of a sensor(s) 154. The mask 10 has a carbon dioxide sensor 154 operably connected to an underside surface of the top wall 28. The carbon dioxide sensor 154, therefore, is positioned in fluid communication with the internal cavity 30. In the exemplary embodiment, the carbon dioxide sensor 154 has Bluetooth communication capabilities to communicate with remote monitors and other equipment. When the mask 10 is positioned on a patient, the sensor 154 can measure exhaled carbon dioxide from the patient or other gases. Measurements from the sensor 154 are wirelessly communicated to associated monitors where additional responders or medical personnel can review accordingly and take further appropriate action for the patient.

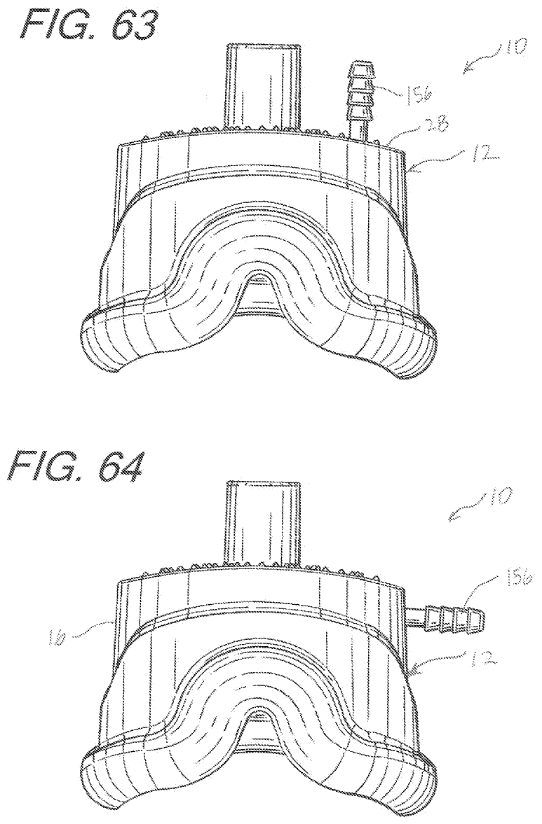

As shown in FIGS. 63-64, the nasal ventilation mask 10 can have an oxygen port 156 on the housing 12 of the mask 10. The oxygen port 156 has a distal end that is in fluid communication with the internal cavity 30 of the mask 10 (It is understood from FIGS. 63-64 that the oxygen port 156 has a passageway in fluid communication with the internal cavity 30.) The oxygen port 156 can further utilize a ribbed structure wherein oxygen supply tubing can be frictionally attached to the oxygen port 156. Once connected a supply of oxygen gas can be delivered into the internal cavity of the mask 10 for further breathing assistance to the patient. FIG. 63 shows the oxygen port 156 positioned on the top wall 28 of the housing 12. FIG. 64 shows the oxygen port 156 positioned on the sidewall 16 of the housing 12. It is understood that the oxygen port 156 could be positioned at other locations of the mask 10.

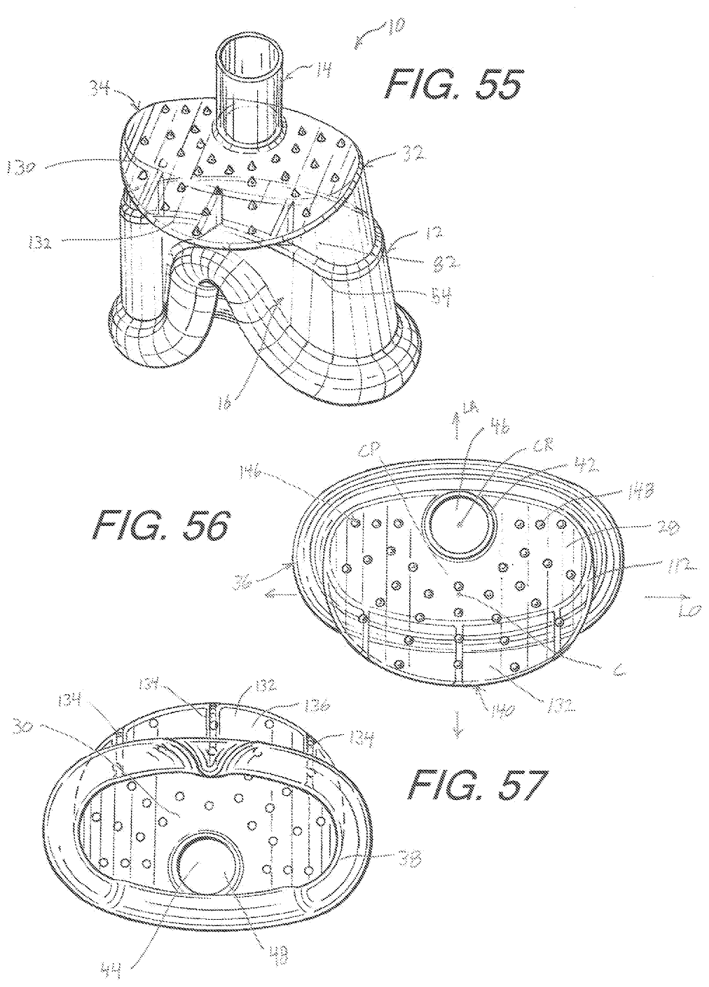

FIGS. 55-60 disclose a nasal ventilation mask according to another exemplary embodiment of the present invention, also designated with the reference numeral 10. The nasal mask 10 of FIGS. 55-60 has similar structures to the nasal mask 10 of FIGS. 3-37. The above descriptions regarding structures and functionality generally apply to this nasal mask 10, and similar structures are referenced with similar reference numerals. Structural features and functionality that are different from the previously-described embodiment will be further described herein and designated with additional reference numerals.

The nasal ventilation mask 10 of FIGS. 55-60 also utilizes the two-piece design having the housing body segment 50 and the connector segment 52. These segments 50,52 have a similar cooperative structure that forms the connection joint 54 as described above. The housing body segment 50 of FIGS. 55-60 is generally the same as the housing body segment 50 of FIGS. 3-37. The connector segment 52 of FIGS. 55-60 has some different and additional features from the connector segment 52 of FIGS. 3-37, which will be further described below.

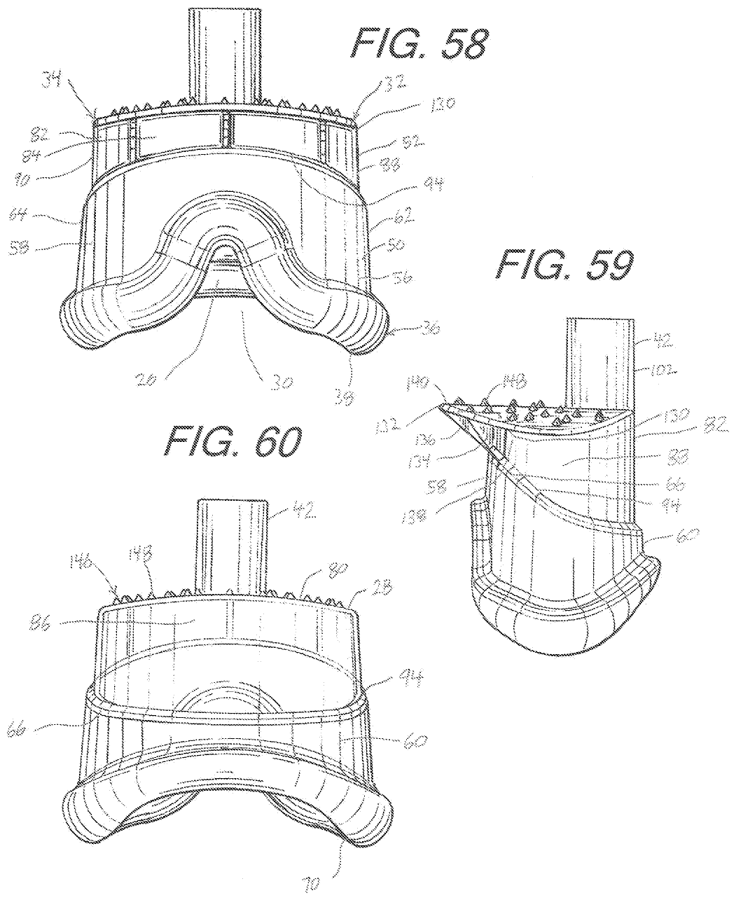

FIGS. 55-60 further show the housing body segment 50. As described above, it is understood that the housing body segment 50 of FIGS. 55-60 is similar in structure to the housing body segment 50 of FIGS. 3-37. As shown in FIGS. 55-60, the housing body segment 50 generally forms a lower portion of the nasal ventilation mask 10, and thus defines, among other structures, the distal end 36 of the mask 10 as well as the sealing member 38. The housing body segment 50 has a lower sidewall segment 56 that forms the lower portion of the sidewall 16 of the housing 12. The lower sidewall segment 56 defines an internal area that will cooperate to form the internal cavity 30. The lower sidewall segment 56 also defines a front wall section 58, a rear wall section 60, a first end wall section 62 and a second end wall section 64. The front wall section 58 contains the nose slot 26 wherein the slot 26 is defined completely in the front wall section 58. As in the previous embodiment, the slot 26 is a portion of the end opening 40 and in communication with the end opening 40. Also as in previous embodiments, the nose slot 26 is generally transverse to the end opening 40. As further shown, the front wall section 58 has a greater height/length dimension than the rear wall section 60. The front wall section 58 has a convex upper perimeter, but with a substantially horizonal central portion (FIG. 58). The lower sidewall segment 56 has a proximal end that defines a first connection surface 66 and facing upwards and away from the distal end 36. It is understood that the first connection surface 66 could take various configurations such as a combination of planar and angled surfaces and any of the configurations described above. The first connection surface 66 has a greater height from the distal end 36 at the front wall section 58 than a height of the first connection surface 66 from the distal end 36 at the rear wall section 60. As can be appreciated from FIGS. 55 and 59, the first connection surface 66 slopes downwardly from the front wall section 58 towards the rear wall section 60. The first connection surface 66 slopes generally along a curved path from the front wall section 58 to the rear wall section 60 on both the first end wall section 62 and the second end wall section 64. The first connection surface 66 has a generally horizontal segment extending from the rear wall section 60. As with the previous embodiment, the first connection surface 66 may define a planar surface and an inclined surface facing into the internal area of the housing body segment 50. The lower sidewall segment 56 has a first thickness t₁ of 0.070 inches in one exemplary embodiment or 0.090 inches in another exemplary embodiment. It is understood that the thickness can be varied. For example, in another exemplary embodiment, the lower sidewall segment 56 could have a thickness of 0.125 inches.

Similar to the embodiment of FIGS. 3-37, the housing body segment 50 defines the distal end 36 and has the sealing member 38 proximate the distal end 36. As can be appreciated from FIGS. 57-60, the sealing member 38 takes the form of the lip member 70, and the housing body segment 50 further has the reinforcing member 68. It is understood that the above description regarding such structural features and functionality applies to the housing body segment 50 of FIGS. 55-60. It is also understood that the housing body segment 50 of FIGS. 55-60 is made from the same materials as discussed above.

FIGS. 55-60 further disclose the connector segment 52. As discussed, the connector segment 52 cooperates with and is operably connected to the housing body segment 50 to form the nasal mask 10 as described in greater detail below. The connector segment 52 generally forms an upper portion of the nasal mask 10. The connector segment 52 has a base portion 80 that generally corresponds to the top wall 28 of nasal mask 10. The connector segment 52 further has an upper sidewall segment 82 that generally depends down from the base portion 80. The base portion 80, or top wall 28, in cooperation with the upper sidewall segment 82 defines an internal area that cooperates with the internal area defined by the housing body segment 50 to define the internal cavity 30. The receiver member 42, described in greater detail below, extends upwards from the top wall 28 and is in fluid communication with the internal cavity 30 when the connector segment 52 is operably connected to the housing body segment 50.

Similar to the lower sidewall segment 56 of the housing body segment 50, the upper sidewall segment 82 defines a front wall section 84, a rear wall section 86, a first end wall section 88 and a second end wall section 90. As further shown, the front wall section 84 has a shorter length dimension than the rear wall section 86. The front wall section 84 has a broad concave lower perimeter. The upper sidewall segment 82 has a proximal end that is integrally connected to the base portion 80 and top wall 28. This connection between the upper sidewall segment 82 and the top wall 28 defines a connection interface 130. The connection interface 130 is at the juncture between the top wall 28 and the upper sidewall segment 82. The connection interface 130 is generally at the outer periphery 112 of the top wall 28. These connections further define the first shoulder 32 and the second shoulder 34 at respective interface areas between the top wall 28 and the upper sidewall segment 82. As can be appreciated from FIGS. 58 and 59, the top wall 28 has a slight curvature across the top wall 28 from proximate the first shoulder 32 to proximate the second shoulder 34. A free depending end of the upper sidewall segment 82 defines the second connection surface 94. As in previous embodiments, the second connection surface 94 also defines an inclined surface and adjacent a planar surface. These surfaces generally correspond and mirror the connection surface of the first connection surface 66.

It is understood that the second connection surface 94 could take various other configurations such as a planar surface, angled surface or a combination thereof. Generally, the second connection surface 92 has a cooperative configuration to be operably connected to the first connection surface 66. The second connection surface 94 is closer in proximity to the top wall 28 at the front wall section 84 than the second connection surface 94 at the rear wall surface 86. In addition, as can be appreciated from FIG. 59, the second connection surface 94 slopes downwardly from the front wall section 84 towards the rear wall section 86. The second connection surface 94 slopes generally along a curved path. The upper sidewall segment 82 has a second thickness t₂ of 0.045 inches in one exemplary embodiment or 0.070 inches in another exemplary embodiment. It is understood that the thickness can be varied. In addition, the top wall 28 has a thickness of 0.070 inches or 0.050 inches. The upper sidewall segment 82 may have the second thickness t₂ that is generally equal to the first thickness t₁ of the lower sidewall segment 56 in this exemplary embodiment. Also, the second connection surface 94 is dimensioned to correspond to the lower sidewall segment 56. Similar to certain of the previous embodiments, the sidewall 16 is generally a straight and conical configuration. In other exemplary embodiments, the sidewall 16 may have a thickness of 0.080 inches or approximately 0.075 inches. The housing body segment 50 may have a part material volume of 0.551 cubic inches. In an exemplary embodiment, the connector segment 52 may have a part material volume of 0.544 cubic inches.

FIGS. 55-60 further show the receiver member 42. Similar to the previous embodiment, the receiver member 42 extends upwards from the top wall 28. The receiver member 42 has the inlet opening 46, internal conduit 44 and outlet opening 48 wherein the receiver member 42 is in fluid communication with the internal cavity 30 of the housing 12. As further shown in FIGS. 55-57 and 59, the receiver member 42 also has an off-set configuration similar to the previous embodiment. With such configuration, additional contiguous, uninterrupted surface area is provided on the top wall 28 for a user or responder to be able to more efficiently press down on the top wall 28 when using the nasal ventilation mask 10 as will be further described below.

As shown in FIG. 55-60, the top wall 28 defines an outer periphery 112 and a center point C generally at the intersection of a longitudinal axis LO and a lateral axis LA associated with the structure of the top wall 28. Thus, the center point C is positioned within the outer periphery 112. A central portion CP of the top wall 28 generally surrounds proximate the center point C wherein the central portion CP is generally circumjacent the center point C. In an exemplary embodiment, the receiver member 42 is positioned remote from the central portion CP. Further, the receiver member 42 is positioned remote from the center point C. In particular, the receiver member 42 is positioned proximate the outer periphery 112 of the top wall 28. Thus, one portion of the receiver member 42 is positioned at the outer periphery 112. As shown in FIG. 59, an outer surface of the cylindrical wall 102 of the receiver member is generally aligned with an outer surface of the upper sidewall segment 82 of the connector segment 52. In such configuration, the inlet opening 46 of the receiver member 42 is considered non-concentric with the end opening 40 of the housing 12. Such positioning provides greater contiguous, uninterrupted surface area on the top wall 28. It is further understood that the receiver member 42 defines a receiver center point CR that is generally at a center of cylindrical wall 102. The receiver center point CR is remote from the center C of the top wall 28 and further remote from the central portion CP.

As further shown in FIGS. 55-57 and 59, the top wall 28 has a portion defining an extension wall member 132. The extension wall member 132 extends from the connection interface 130 proximate the front wall section 84 of the upper sidewall segment 82. The extension wall member 132 provides additional surface area of the top wall 128 beyond where the upper sidewall segment 82 meets the top wall 28 at the connection interface 130. In an exemplary embodiment, the extension wall member 132 is an integral structure with the top wall 28 and upper sidewall segment 82. The extension wall member 132 may be considered to be a cantilevered structure adjacent the connection interface 130. A support member 134 is positioned between an underside surface 136 of the extension wall member 132 and an outer surface 138 of the upper sidewall segment 82. In an exemplary embodiment, the support member 134 is a wedge-shaped member having an inclined free end. Also in an exemplary embodiment, the support member 134 comprises a plurality of support members 134 spaced along the underside surface 136 of the extension wall member 132. Three spaced support members 134 may be utilized in a further exemplary embodiment. The support members 134 assist in stabilizing the extension wall member 132 that overhangs the upper sidewall segment 82 as a responder may apply force F via hand thumb/finger pressure on the extension wall member 132. As further shown in FIGS. 55-57, the extension wall member 132 has a distal edge 140 having a curved configuration. As also shown in FIG. 59, the extension wall member 132 has a length that extends beyond the overall footprint of the mask 10 and thus, beyond the nose slot 26 and outer periphery of the reinforcing member 68 of the housing 12.

As further shown in FIGS. 55-60, the base portion 80 or top wall 28 of the connector segment 52 has a gripping member 146 in the form of a plurality of protrusions 148. The protrusions 148 extend upwards from the top wall 28 and extension wall member 132. The protrusions 148 are generally cone-shaped and are dispersed around the top wall 28 including on the extension wall member 132. The protrusions 148 can be randomly placed on the top wall 28 and extension wall member 132 and can also be organized in a plurality of spaced apart linear segments emanating from around the receiver member 42. The protrusions 148 are dimensioned to provide tactile feel and an anti-slip finger gripping feature for responder convenience for forcing and holding the mask firmly against the patient's face. It is understood that the height of the protrusions 148 can vary but have a height to be engaged by a user and further to provide tactile feedback including to a user wearing protective gloves.

FIGS. 55-60 further shown the receiver member 42. In certain medical standards for respiratory equipment/conical connectors, a 1.432 degree internal diameter taper is specified. An external approximate 1.5 degree taper (draft angle) on the receiver member 42 is utilized for reliable ejection of the part during the molding process. A distal end forming the outlet opening of the receiver member 42 is formed at approximately 0.030 inches. With such parameters, the wall thickness of the receiver member 42 proximate the top wall 28 is approximately 0.074 inches. Modifying the draft angle can result in other desired wall thicknesses. As previously discussed, the receiver member 42 has dimensions to conform to International Standard specifications for socket connectors for respiratory equipment.

As can be appreciated from FIGS. 55 and 58-60, the housing body segment 50 is operably connected to the connector segment 52 to form the nasal ventilation mask 10. When operably connected, the first connection surface 66 and the second connection surface 94 abut and are joined together to form the connection joint 54. The upwardly facing first connection surface 66 of the housing body segment 50 confronts the downward facing second connection surface 94 of the connector segment 52. As in the previous exemplary embodiment, the connection joint 54 is formed via an over-molding injection process wherein the segments 50,52 are joined together. For example, the connector segment 52 can be injection molded in a first mold assembly initially and after completion, placed into another mold assembly. The housing body segment 50 can then be injected molded, or over-molded onto the connector segment 52 resulting in the configuration described above and wherein a chemical bond is formed at the connection joint 54 between the materials of the housing body segment 50 and the connector segment 52. In addition, more complex mold assemblies can be used wherein the housing body segment 50 is molded sequentially during the same overall process as the molding of the connector body segment 52. Other connection methods are also possible such as adhesive bonding or other chemical bonding. Interlocking mechanical connections can also be utilized. Combinations of the above-described connection methods can also be utilized. It is further understood that in a two-shot molding process or over-molding process in forming the housing body segment 50 and connector segment 52, a chemical bond can also be formed between the segments 50,52. A chemical bond is formed between the two segments 50,52 to form the connection joint 54.

FIGS. 55-60 further show the connection joint 54 formed between the housing body segment 50 and the connector segment 52. The connection joint 54 generally has a contoured path around the circumferential sidewall 16. The contoured path provides enhanced operability of the mask 10. The connection joint 54 is more proximate to the top wall 28 at the front wall section 18 of the mask 10 than at the rear wall section 20 of the mask 10. In this exemplary embodiment, the connection joint 54 has a profile in a curved, slanted configuration. In particular, the connection joint 54 generally slopes downwardly along a curved path at the first end wall section 22 and the second end wall section 24 from the front wall section 18 towards the rear wall section 20. Thus, as can be appreciated from FIG. 49, at the first end wall section 22 and at the second end wall section 24, the connection joint 54 has the downwardly curved configuration.

The longer rear wall 86 of the connector segment 52 provides additional rigidity as the connector segment 52 is formed from the more rigid material. The additional rigid material is positioned at the lip area of the patient to minimize local flaring of the mask 10. Thus, the housing body segment 50 has a lesser dimension at the rear wall section 60 corresponding to the area configured to engage proximate the upper lip of the patient, and further has less flex at this area where flaring is undesirable. This configuration still allows the side contours of the mask 10 to flex or flare in a controlled manner to allow for variations in facial contours of patients. Thus, more portions of the nasal mask 10 are formed of the rigid material at strategic locations while the portions of the mask 10 that require flexibility for forming a good airtight seal against the patient's skin are formed of the more flexible material. The curved connection joint 54 achieves these characteristics and it is understood that the connection joint 54 could further have other configurations. The connection joint 54 could have a different contoured configuration or other profile that achieves the similar results to provide an optimal seal against the skin of the patient.

Once formed, the nasal mask 10 of FIGS. 55-60 can also be used with the manual resuscitator bag assembly 1. The nasal mask 10 is used in the same fashion as described above wherein emergency responders press the mask 10 against the face of the patient as can be appreciated from FIGS. 38-43. The supply of breathing gas can be delivered to the patient. The above descriptions regarding operation and functionality apply to the nasal mask 10 of FIGS. 55-60.

Thus, it is understood that the nasal ventilation mask 10 of FIGS. 55-60 is positioned on a patient similar to FIGS. 38-48. Similar as described above, the emergency responder can apply force F on the mask towards the patient's face to further enhance the seal. The emergency responder can use, for example, a thumb, or a thumb and finger, or other fingers, to press on the top wall 28 and/or first shoulder 32 and the second shoulder 34 as well as the extension wall member 132. As the shoulders 32,34 provide a portion of a planar, platform-type surface that is rigid, the medical worker can easily apply any requisite force F to the mask 10 towards the patient's face to further enhance the seal (FIG. 40). As described above, the receiver member 42 has the off-set configuration wherein the receiver member 42 is positioned generally proximate the outer periphery 112 of the top wall 28. Thus, when the mask 10 is properly positioned on the patient, the receiver member 42 is positioned closer to the upper lip of the mouth of the patient. The configuration thus provides more specific contiguous, uninterrupted surface area on the top wall 28 more towards the rear wall section of the mask 10. The responder can then apply pressure more efficiently at this specific surface area so that force can be more efficiently applied more proximate the upper lip of the mouth of the patient which allows the responder to achieve a better seal directly underneath the nose of the patient. This configuration thus allows the responder's thumb/finger to be closer towards the mouth of the patient, which further allows for other responder fingers to reach under and cradle a jawline of the patient to achieve additional support when applying force to further enhance the sealing capability of the mask 10. This is particularly beneficial for responders having smaller-sized hands. Further in this configuration, the top wall 28 has more contiguous, uninterrupted surface area capable of being engaged by the responder for applying force on the mask 10 to create a good seal. In addition, the extension wall member 132 provides additional surface area associated with the top wall 28, generally opposite the area where the receiver member 42 is located, for a responder to press and provide force F on the mask 10 to create a more enhanced and tight seal.

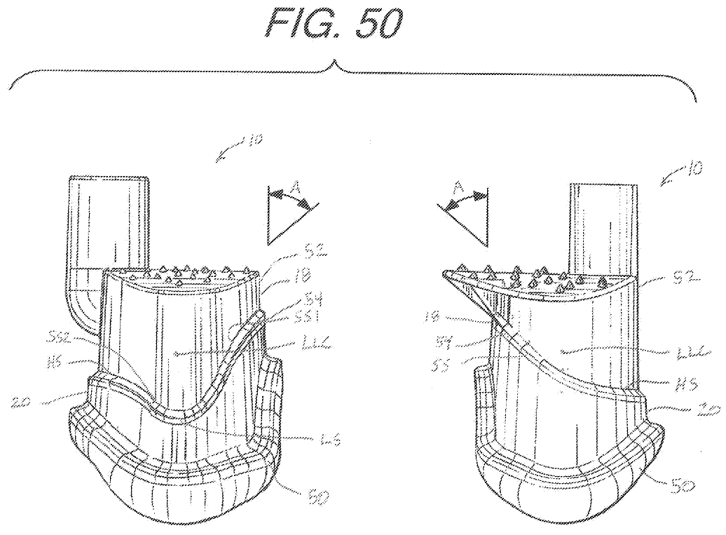

As discussed, the exemplary embodiments of FIGS. 3-37 and FIGS. 55-60 utilize a two-piece design forming the connection joint 54. The connection joint 54 is formed between the housing body segment 50 and the connector segment 52. As discussed, the connection joint 54 of FIGS. 3-37 and FIGS. 55-60 have a profile in a generally curved-slanted configuration. FIG. 50 shows comparison side elevation views of the nasal ventilation masks 10 of FIGS. 3-37 (left-side mask 10) and FIGS. 55-60 (right-side mask 10). As shown in FIG. 50, the connection joint 54 of the left-side mask 10 extends from the front wall section 18 towards the rear wall section 20 along a curved line or path, or downwardly sloped line or path. It is understood the housing body segment 50 and the connector segment 52 have the corresponding curved-slanted profiles that mate to form the connection joint 54 when operably connected. Thus, at proximate a lateral/longitudinal central portion LLC at respective end wall sections 22,24 of the mask 10, the mask is formed of the more rigid material of the connector segment 52. The lateral/longitudinal central portion LLC is an area on each of the first end wall section 22 and second end wall section 24 located at generally an intersection of a midpoint of a lateral dimension from the front wall section 18 to the rear wall section 20 of the mask 10 and of a midpoint of a longitudinal dimension of an overall height of the mask 10 from the distal end 36 to the inlet opening 46 of the receiver member 42. This can be appreciated from the left-side mask 10 in FIG. 50. With the curved profile, the more flexible housing body segment 50 is positioned below the lateral/longitudinal central location LLC while the connector segment 52 having more rigid material occupies the lateral/longitudinal central location LLC. This is further appreciated from the comparison of side elevation views in FIG. 50. In the right-side mask 10 having another curved connection joint 54, the connection joint 54 is also generally below the lateral/longitudinal central portion LLC of the end wall sections 22,24 of the mask 10 wherein the connection joint 54 passes below the lateral/longitudinal central portion LLC of the end wall sections 22,24 of the mask 10.

With the lower positioning of the connection joint 54 at the respective end wall sections of the exemplary embodiments shown in FIG. 50, the mask 10 is formed of the more rigid material of the connector segment 52 at a larger area of the first end wall section 22 and second end wall section 24 of the of the mask 10. This configuration assists in further stabilizing the mask 10 and resists undue flaring when pressing the mask 10 against a patient's skin during use. Thus, the configuration provides for more controlled flexibility of the mask 10 at the end wall sections 22,24 to accommodate varying patient facial contours while still providing for an enhanced seal against the face of the patient. Such configuration further provides material cost-saving benefits. In one exemplary embodiment, the curved slanted profile of the connection joint 54 is the result of a conic profile that travels linearly from one side of the mask body to the other side of the mask body in a path generally normal to a central plane of symmetry of the mask body. As discussed, the curved or downwardly sloped profile of the connection joint 54 could take other forms as well. The connection joint 54 could also be a radial curve or some arc of a circle. It is understood that the housing body segment 50 and the connector segment 52 have the corresponding and confronting curved slanted profiles. It is further understood that curved connection joint 54 of the right-side mask 10 also provides a greater amount of rigid material on the connection segment 52 on the end wall sections 22,24 (as opposed, for example, of a straight annular connection joint 54). The curved/slanted connection joint 54 provides more enhanced functionality over the linear slanted connection joint 54.

Referring to FIG. 50, with the curved slanted connection joint 54 shown in the left-side mask 10, the connection joint 54 may be considered to have a generally conic curve profile at the end wall sections 22,24 of the mask 10, but also an additional sloped segment and a lowermost segment as described above. As shown in the side elevation view, the connection joint 54 profile extends, or swings, or slopes downwardly from approximately an angle A of approximately 30° from a vertical axis at the front of the mask 10. The connection joint 54 continues sloping downwardly to a lowermost segment LS. The connection joint 54 has an approximately horizontal segment HS generally at the rear wall section 20 of the mask 10. The connection joint 54 has a second sloped segment SS2 from the horizontal segment HS that meets with the lowermost segment LS. Thus, from the side elevation view, the connection joint 54 has the downwardly sloped profile from the front wall section 18 of the mask 10 and towards the rear wall section 20 of the mask 10. This profile may be considered to look like or resemble a ski slope shape. Thus, at the end wall sections 22,24, the connection joint 54 has the first sloped segment SS1, as well as the second sloped segment SS2 and a substantially horizontal segment HS. The first sloped segment SS1 meets with the second sloped segment SS2 at the lowermost segment LS. As is further appreciated from the side elevation view, the housing body segment 50 has lesser dimensions on the end wall sections 22,24 wherein the mask 10 has more rigid material of the connector segment 52 on the end sections 22,24. Overall, the connection joint 54 has a concave configuration at the front wall section 18 and a generally horizontal configuration at the rear wall section 20 while having the curved slanted configuration as described on the end wall sections 22,24. It is further understood that the curved slanted configurations of the connection joint 54 at the end wall sections 22,24 have the downwardly sloped segments SS1,SS2 and a substantially horizontal segment HS more proximate to the rear wall section 20.

Referring further to FIG. 50, with the curved slanted connection joint 54 shown in the right-side mask 10, the connection joint 54 may be considered to have a generally conic curve profile at the end wall sections 22,24 of the mask 10. As shown in the side elevation view, the connection joint 54 profile extends, or swings, or slopes downwardly from approximately an angle A of approximately 30° from a vertical axis at the front of the mask 10. The connection joint 54 continues sloping downwardly to an approximately horizontal profile proximate the rear wall section 20 of the mask 10. Thus, from the side elevation view, the connection joint 54 has the downwardly sloped profile from the front wall section 18 of the mask 10 to the rear wall section 20 of the mask 10. This profile may be considered to look like or resemble a ski slope shape. Thus, at the end wall sections 22,24, the connection joint 54 has a sloped segment SS and a substantially horizontal segment HS. As is further appreciated from the side elevation view, the housing body segment 50 has lesser dimensions on the end wall sections 22,24 wherein the mask 10 has more rigid material of the connector segment 52 on the end sections 22,24. Overall, the connection joint 54 has a concave configuration at the front wall section 18 and a generally horizontal configuration at the rear wall section 20 while having the curved slanted configuration as described on the end wall sections 22,24. It is further understood that the curved slanted configurations of the connection joint 54 at the end wall sections 22,24 have the downwardly sloped segment and a substantially horizontal segment more proximate to the rear wall section 20.

The nasal ventilation masks 10 of FIGS. 3-37 and 55-60 utilize many of the same features described herein such as the inwardly curled lip member 70 and the reinforcing member 68. It is understood that the housing body segment 50 in certain exemplary embodiments may have a material part volume of approximately 0.462 cubic inches. Also in certain exemplary embodiments, the connector body segment 52 may have a material part volume of approximately 0.396 cubic inches. The housing body segment 50 may also be formed from the materials described above and further be selected to have a Shore A durometer in the range of 30-40 and, in particular, a Shore A hardness of 30-32 in further exemplary embodiments. It is also contemplated that the overall height of the mask 10 can be increased such as by increasing the height of the shoulders 32,34 approximately 0.125 inches to accommodate patients with varying nose profiles.

It is further understood that in any of the exemplary embodiments described herein, it is desirable for materials of the mask 10 to be as transparent as possible. It is further understood that materials may also be translucent. For example, in two-piece embodiments, the housing body segment 50 may have translucent properties and the connector segment 52 may have transparent properties.

As described herein, the various exemplary embodiments of the ventilation mask can be made of the various material described. In one exemplary embodiment described, the housing body segment is made from a more flexible material and the connector segment is made from a more rigid material. In a particular exemplary embodiment, the housing body segment is made from a thermoplastic elastomer material and the connector segment is made from an acrylonitrile butadiene styrene material. It is understood that additional materials can also be utilized. In an additional exemplary embodiment, the housing body segment or in particular, the sealing member could be made from a silicone material. It is further understood that the sealing member could be an air-filled silicone member. It is also understood that other materials are also possible such as thermoplastic elastomer materials or generally soft materials. Thus, the sealing member, which engages and abuts the skin of the face of the patient, could be made from silicone, while other parts of the mask could be made from other materials wherein the connector segment utilizes the offset configuration of the receiver member as described herein.

Multiple embodiments of the nasal ventilation mask are disclosed herein. It is understood that in main exemplary embodiments, the ventilation mask is designed to be placed over a nasal airway of a patient wherein the oral cavity of the patient remains unobstructed as described above. It is understood that many of the features of the specific nasal ventilation mask can be used with a ventilation mask designed to be placed over both the nasal airway and oral airway of a patient.

It is understood that multiple exemplary embodiments are disclosed and described in the present application. The various exemplary embodiments share certain features and also utilize different features in certain embodiments. It is understood that the various features can be used in various combinations in yet further embodiments.

The ventilation mask or nasal ventilation mask provides several benefits. The sealing structures associated with the mask provide enhanced pneumatic seals between the patient's skin and the mask. The seal is airtight to maintain air pressure within the internal cavity of the mask to efficiently deliver a supply of breathing gas to the patient. The inwardly curled lip provides an enhanced seal in response to force being applied to the mask against the skin of the patient. It is understood that the seal is formed around the fully periphery of the end opening of the mask including the slot wherein the seal is formed at the sides of the nose and the dorsal base of the nose. The nasal ventilation mask has a two-piece construction in certain exemplary embodiments which assists in the mask having rigidity where desired. The mask has flexible portions where needed such in the housing body segment and sealing member and more rigid properties in the connector segment. In other embodiments, the nasal mask has one-piece construction that provides for enhanced operation and can be manufactured efficiently. It is understood that the reinforcing member positioned around the periphery of the housing body segment and proximate the distal end, provides additional stiffening to the body segment. This helps to minimize any undue buckling or deformation when personnel may apply force to the mask 10. In this configuration, the reinforcing member and lip member provide an integrated reinforcing structure that minimizes potential floppiness in the mask. Such integration provides a cleaner, enhanced design as well for allowing more efficient resin flow in the mold when forming the component. The mask further utilizes gripping members to assist in providing tactile feel to medical personnel using the mask on a patient. The structural profile of the mask also provides benefits. The generally oval footprint of the end opening and size/height of the slot to accommodate the patient's nose are dimensioned to provide an enhanced seal against the patient's face. The height of the slot extends only to the dorsal base of the nose rather than extending to a dorsal bridge of the nose closer to eyes of the patient in certain embodiments. This minimizes the overall linear surface area of the sealing member required to form the seal. The transverse relationship of the nose slot with respect to the end opening further assists with these benefits. With such structural configuration, a single-sized mask can achieve an airtight seal on multiple sized patients and having varying facial contours. The mask can be successfully used on an infant, child or an adult. It is further understood that masks of different sizes can further be employed to accommodate additional patients of varying age and size. In addition, the positioning of the receiver member more proximate an outer periphery of the top wall or beyond the outer periphery of the top wall provides for enhanced sealing capabilities by a responder. This configuration allows a responder to apply force more directly proximate the upper lip of the patient and further provides more contiguous, uninterrupted surface area for applying force. The extension wall member also provides additional surface area for applying force. This configuration also provides ergonomic advantages wherein a responder can apply force using a single hand. Such configuration further allows a responder to reach under a jawline of a patient with fingers to provide additional support when applying force thus achieving an enhanced seal. This configuration further makes this easier to achieve for responders having smaller hands.

While the invention has been described in its preferred embodiments, it is to be understood that the words which have been used are words of description rather than limitation and that changes may be made within the purview of the appended claims without departing from the true scope and spirit of the invention in its broader aspects.

What is claimed is:

1. A ventilation mask for supplying breathing gas to an airway of a patient, the ventilation mask comprising:
a housing defining an internal cavity and having a peripheral end defining an end opening, the end opening being in fluid communication with the internal cavity, the end opening configured to be positioned over an airway on a face of a patient, the housing further defining a connector segment having a receiver member, the connector segment defining a top wall defining an outer periphery wherein the receiver member has a portion extending past the outer periphery, the receiving member having an opening in fluid communication with the internal cavity;
a sealing member operably connected to the housing proximate the end opening, the sealing member configured to abut the face of the patient, and
wherein the receiver member is configured to be operably connected to a supply of breathing gas.

2. The ventilation mask of claim 1 wherein the top wall defines a central portion, the receiver member extending from the top wall at a location remote from the central portion.

3. The ventilation mask of claim 1 wherein the housing defines an upper sidewall segment wherein the receiver member has a portion extending from the top wall and a portion extending from the upper sidewall segment.

4. The ventilation mask of claim 1 wherein the housing defines a front wall section and a rear wall section depending from the top wall, wherein the receiver member has a portion extending from the top wall and a portion extending from the rear wall section.

5. The ventilation mask of claim 1 wherein the housing defines a front wall section and a rear wall section depending from the top wall, the housing defining a nose slot in the front wall section, wherein the receiver member extends from the top wall proximate the rear wall section and generally opposite the nose slot.

6. The ventilation mask of claim 1 wherein the housing defines a front wall section and a rear wall section depending from the top wall, the housing defining a nose slot in the front wall section, wherein the receiver member defines a cylindrical wall wherein a portion of the cylindrical wall extends from the top wall and a portion of the cylindrical wall extends from the rear wall section and generally opposite the nose slot.

7. The ventilation mask of claim 1 wherein the top wall defines an outer periphery and a center point location within the outer periphery, wherein the receiver member extends from the top wall at a location remote from the center point location and proximate the outer periphery.

8. The ventilation mask of claim 1 wherein the opening of the receiver member is not concentric with the end opening.

9. The ventilation mask of claim 1 wherein the sealing member has a reinforcing member extending from the housing, and the housing defines an upper sidewall segment depending from the top wall, the receiver member has a portion extending from the top wall and a portion extending from the upper sidewall segment that is generally aligned with an end of the reinforcing member.

10. The ventilation mask of claim 1 wherein the receiver member is integral with the connector segment.

11. The ventilation mask of claim 1 wherein the sealing member extends towards the internal cavity.

12. The ventilation mask of claim 1 wherein the sealing member forms an inwardly curled lip member extending into the internal cavity, the lip member having an outer surface configured to abut the face of the patient.

13. The ventilation mask of claim 12 wherein the lip member has a proximal end connected at the peripheral end, the lip member having a distal end, the lip member having a thickness being tapered towards the distal end.

14. The ventilation mask of claim 12 wherein the housing defines an internal surface wherein an imaginary line extends from the internal surface past the end opening, wherein a distal end of the lip member extends past the imaginary line.

15. The ventilation mask of claim 12 wherein an outer surface of the inwardly curled lip has a convex shape and is smooth and uninterrupted.

16. The ventilation mask of claim 1 wherein the housing has a reinforcing member extending therefrom proximate the end opening.

17. The ventilation mask of claim 16 wherein the reinforcing member has an outer end, wherein the sealing member defines an inwardly curled lip member having a proximal end depending from the outer end of the reinforcing member.

18. The ventilation mask of claim 17 wherein the housing defines an internal surface wherein an imaginary line extends from the internal surface past the end opening, wherein a distal end of the lip member extends past the imaginary line.

19. The ventilation mask of claim 1 wherein the housing further defines a housing body segment operably connected to the connector segment defining a circumferential connection joint.

20. The ventilation mask of claim 19 wherein the housing defines a front wall section opposite a rear wall section and a first end wall section extending between the front wall section and the rear wall section, wherein the connection joint extends downwardly along a contoured path on the first end wall section from the front wall section towards the rear wall section.

21. The ventilation mask of claim 20 wherein the connection joint on the first end wall section has a curved segment and a substantially horizontal segment proximate the rear wall section.

22. The ventilation mask of claim 19 wherein the housing body segment defines a lower sidewall segment and the connector segment defines an upper sidewall segment, the upper sidewall segment positioned inwardly offset from the lower sidewall segment.

23. The ventilation mask of claim 22 wherein the lower sidewall segment has a first thickness and the upper sidewall segment has a second thickness, the first thickness being greater than the second thickness.

24. The ventilation mask of claim 19 wherein the connection joint is a formed by confronting connection surfaces between the housing body segment and the connector segment wherein a chemical bond is formed between the materials of the housing body segment and the connector segment.

25. The ventilation mask of claim 21 wherein the housing body segment is of a thermoplastic elastomer material.

26. The ventilation mask of claim 21 wherein the connector segment is of an acrylonitrile butadiene styrene material.

27. The nasal ventilation mask of claim 1 wherein the top wall has a gripping member thereon.

28. The ventilation mask of claim 27 wherein the gripping member comprises a plurality of protrusions.

29. The ventilation mask of claim 1 wherein the top wall has a curved configuration from a first top wall end to a second top wall end.

30. The ventilation mask of claim 1 wherein the receiver member extends generally perpendicular from the top wall wherein when the mask is configured to abut the face of the patient, the receiver member is configured to be generally perpendicular to the face of the patient.

31. A ventilation mask for supplying breathing gas to an airway of a patient, the ventilation mask comprising:
a housing defining an internal cavity and having a peripheral end defining an end opening, the end opening being in fluid communication with the internal cavity, the end opening configured to be positioned over an airway on a face of a patient,
the peripheral end of the housing having an inwardly curled lip extending towards the internal cavity, the inwardly curled lip configured to abut the face of the patient and deflect towards the internal cavity,
the housing further defining a front wall section, a rear wall section and opposite end wall sections extending between the front wall section and the rear wall section, the housing comprising a housing body segment operably connected to a connector segment defining a circumferential connection joint around the housing, wherein on the opposite end wall sections the connection joint has segments sloping downwardly along a curved path from the front wall section towards the rear wall section, and wherein further on the opposite end wall sections, the connection joint has horizontal segments at the rear wall section and extending from the rear wall section towards the front wall section and joining the segments sloping downwardly, and the connector segment defining a top wall defining a central portion, the connector segment further having a receiver member extending from the wall at a location remote from the central portion, the receiving member having an inlet opening in fluid communication with the internal cavity, the receiving member configured to be operably connected to a supply of breathing gas.

32. A nasal ventilation mask for supplying breathing gas to an airway of a patient, the nasal ventilation mask comprising:
a housing having a front wall section, a rear wall section and opposite end wall sections extending between the front wall section and the rear wall section cooperatively defining an internal cavity and having a peripheral end defining an end opening and further defining nose slot in the front wall section, the nose slot being in fluid communication with the end opening, the end opening being in fluid communication with the internal cavity, the end opening configured to be positioned over an airway on a face of a patient with the nose slot configured to be positioned over a nose of the patient,
the housing further defining a connector segment defining a top wall defining an outer periphery, the connector segment further having a receiver member defining an inlet opening in communication with the internal cavity wherein the receiver member extends from the top wall and the rear wall section generally opposite the nose slot in the front wall section wherein a portion of the receiver member extends past the outer periphery of the top wall;
a sealing member operably connected to the housing proximate the end opening, the sealing member configured to abut the face of the patient, and
wherein the receiver member is configured to be operably connected to a supply of breathing gas.

33. A nasal ventilation mask for supplying breathing gas to a nasal airway of a patient, the nasal ventilation mask comprising:
a housing defining an internal cavity and comprising a housing body segment of flexible material operably connected to a connector segment of rigid material defining a circumferential connection joint,
the housing body segment defining a distal end defining an end opening in fluid communication with the internal cavity, the housing body segment further defining a nose slot on a side of the housing wherein the noses slot is generally transverse to the end opening, the slot being in fluid communication with the end opening, the distal end having a sealing member positioned around the end opening and slot, the sealing member forming an inwardly curled lip member extending into the internal cavity,
the connector segment defining a top wall connected to an upper sidewall segment, the top wall defining an outer periphery and having a plurality of rigid protrusions extending from the top wall, the connector segment further having a receiver member defining an inlet opening in communication with the internal cavity wherein the receiver member extends from the top wall and the upper side segment generally opposite the nose slot wherein a portion of the receiver member extends past the outer periphery of the top wall, wherein the top wall defines a contiguous uninterrupted surface area across a central portion of the top wall for applying a pressing force thereon,
wherein the top wall is configured to be pressed by a responder towards a face of the patient wherein the lip member is configured to engage the face of the patient and deflect into the internal cavity and configured to create a seal between the face of the patient and the lip member, and wherein the slot is dimensioned to be positioned proximate a dorsal base of a nose of the patient,
wherein an inflatable bag of a manual resuscitator bag assembly is configured to be operably connected to the inlet opening of the receiver member of the connector segment such that breathing gas is configured to be delivered to the nasal airway of the patient.

34. A ventilation mask for supplying breathing gas to an airway of a patient, the ventilation mask comprising:
a housing defining an internal cavity and having a peripheral end defining an end opening, the end opening being in fluid communication with the internal cavity, the end opening configured to be positioned over an airway on a face of a patient, the housing further having a top wall and a sidewall depending from the top wall at a connection interface, the top wall having an extension wall member extending from the connection interface, the housing having a receiver member extending from the top wall, the receiving member having an opening in fluid communication with the internal cavity;
a sealing member operably connected to the housing proximate the end opening, the sealing member configured to abut the face of the patient, and
wherein the receiver member is configured to be operably connected to a supply of breathing gas.

35. The ventilation mask of claim 33 wherein the top wall defines a central portion, wherein the receiver member extends from the top wall at a location remote from the central portion.

36. The ventilation mask of claim 34 wherein the top wall defines an outer periphery wherein the receiver member is positioned on the top wall at the outer periphery.

37. The ventilation mask of claim 33 further comprising a plurality of support members positioned between an underside surface of the extension wall member and the sidewall.

38. The ventilation mask of claim 33 wherein the top wall has a plurality of protrusions extending therefrom.
